(19) United States
(12) Patent Application Publication
(10) Pub. No.: US 2022/0169606 A1
(43) Pub. Date: Jun. 2, 2022

(54) COMPOSITIONS AND COMPOUNDS FOR BIOANALYSIS

(71) Applicant: Small Pharma Ltd., London (GB)

(72) Inventors: Peter Rands, London (GB); Ellen James, London (GB); Tiffanie Benway, London (GB)

(73) Assignee: Small Pharma Ltd., London (GB)

(21) Appl. No.: 17/108,938

(22) Filed: Dec. 1, 2020

(51) Int. Cl. C07D 209/16; G01N 33/94

(52) U.S. Cl. CPC C07D 209/16; G01N 33/94

(57) ABSTRACT

The present invention relates to compositions comprising compounds of formula I and II. These compositions have uses in the treatment of psychiatric or neurological disorders. Varying the relative amounts of the compounds of formula I and II within the compositions is expected to modulate the therapeutic effect. Related compounds of formula Ill comprise d₃-monomethylamino or d₆-dimethylamino groups and are useful internal standards for quantifying the amount of a target compound, such as an analogous compound comprising protio-monomethylamino or protio-dimethylamino groups. Thus, the present invention also relates to compounds of formula Ill and their uses as internal standards in assays for quantifying the amount of a target compound in a sample. A method of quantifying the amount of a target compound in a sample by adding a known amount of a compound of formula Ill to the sample is also included.

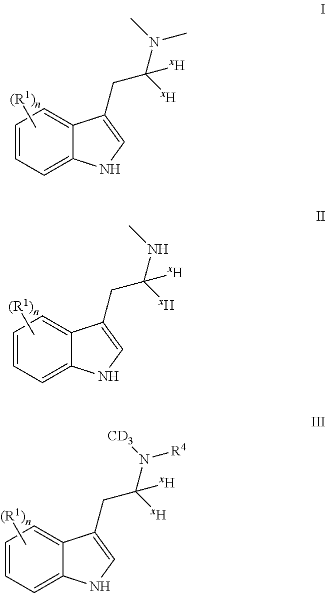

### FIELD

The present invention relates to compositions comprising compounds of formulae I and II. These compositions have uses in the treatment of psychiatric or neurological disorders. Varying the relative amounts of the compounds of formulae I and II within the compositions is expected to modulate the therapeutic effect of the composition. Related compounds of formula III comprise d₃-monomethylamino or d₆-dimethylamino groups and are useful internal standards for quantifying the amount of a target compound, such as an analogous compound comprising protio-monomethylamino or protio-dimethylamino groups. Thus, the present invention also relates to compounds of formula III and their uses as internal standards in assays for quantifying the amount of a target compound in a sample. A method of quantifying the amount of a target compound in a sample by adding a known amount of a compound of formula III to the sample is also included.

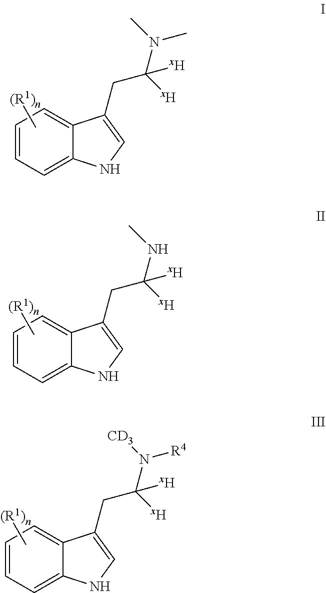

### BACKGROUND

Classical psychedelics have shown preclinical and clinical promise in treating psychiatric disorders (Carhart-Harris and Goodwin, Neuropsychopharmacology 42, 2105-2113 (2017)). In particular, psilocybin has demonstrated significant improvement in a range of depression and anxiety rating scales in randomised double blind studies (Griffiths et al. Journal of Psychopharmacology, 30(12), 1181-1197 (2016)). Efficacy of psilocybin has been shown in depression (R. L. Carhart-Harris et al., Psychopharmacology, 2018, 235, 399-408), end of life anxiety (R. R. Griffiths et al., J. Psychopharmacol., 2016, 30, 12, 1181-1197) and addiction (M. W. Johnson, A. Garcia-Romeu and R. R. Griffiths, Am. J. Drug Alcohol Abuse, 2017, 43, 1, 55-60), and is currently being investigated for several other mental health disorders that are rooted in psychologically destructive patterns of thought processing (Anorexia Nervosa: NCT #NCT04052568).

5-methoxy-N,N-dimethyltryptamine (5-MeO-DMT) is an endogenous tryptamine found in human blood, urine, and spinal fluid (S. A. Barker, E. H. McIlhenny and R. Strassman, Drug Test. Anal., 2012, 4, 7-8, 617-635; F. Benington, R. D. Morin and L. C. Clark, J. Med. Sci., 1965, 2, 397-403; F. Franzen, and H. Gross, Nature, 206, 1052; R. B. Guchhait., J. Neurochem., 1976, 26, 1, 187-190), and has been shown to exhibit protective and therapeutically relevant effects. Anti-depressant properties have been shown in rodents administered 5-MeO-DMT (M. S. Riga et al., Neuropharmacology, 2017, 113, A, 148-155). In addition, a high number of users of 5-MeO-DMT, having ingested it in different forms, reported therapeutic effects attributed to its use, including improved post-traumatic stress disorder, depression and anxiety (A. K. Davis et al., J. Psychopharmacol., 2018, 32, 7, 779-792). 5-MeO-DMT has also exhibited the potential to treat substance abuse disorders (V. Dakic et al., Sci. Rep., 2017, 7, 12863).

N,N-dimethyltryptamine (DMT) is also understood to hold therapeutic value as a short-acting psychedelic. A review of research into the biosynthesis and metabolism of DMT in the brain and peripheral tissues, methods and results for DMT detection in body fluids and the brain, new sites of action for DMT, and new data regarding the possible physiological and therapeutic roles of DMT is provided by S. A. Barker in Front. Neurosci., 12, 536, 1-17 (2018). In this review, DMT is described as having a possible therapeutic role in the treatment of depression, obsessive-compulsive disorder, and substance abuse disorders.

N-Methyltryptamine (NMT) is often extracted together with DMT and 5-MeO-DMT from the bark, shoots and leaves of several plant genera. NMT is reported to have psychedelic properties: smoking NMT gives “visuals” at 50-100 mg, with a duration of 15-30 seconds (Shulgin, A. and Shulgin, A., 2002, THIKAL: the continuation, Transform Press).

DMT and its substituted analogues, such as 5-MeO-DMT, are inactivated through a deamination pathway mediated by monoamine oxidases (MAO). MAOs are found in most cell types of the body. Consequently, DMT and its substituted analogues, such as 5-MeO-DMT are often administered with MAO inhibitors (MAOIs) to prevent inactivation of the compounds before they have reached their target site in the body, allowing for a prolonged and increased exposure to the compound. However, MAOIs can cause high blood pressure when taken with certain foods or medications, thus the use of MAOIs by a patient typically requires the patient to restrict their diet and avoiding some other medications.

In light of the therapeutic potential of substituted dialkyltryptamines such as 5-MeO-DMT, there remains a need in the art for such compounds with improved bioavailability, extended and/or modified pharmacokinetics and/or modified pharmacodynamics, in particular for the development of clinically applicable psychedelic drug substances to assist psychotherapy. The present invention addresses this need.

Bioanalysis, i.e. the accurate measure of the concentration of a drug (such as DMT, psilocybin and 5-MeO-DMT) in bodily fluids, is necessary in drug development, e.g. to determine the pharmacokinetics of the drug and assess how best to develop the drug. The presence of a drug within a sample is typically detected using mass spectrometry (MS), typically electron spray ionisation (ESI), often in conjunction with high performance liquid chromatography (HPLC), which is used to separate the drug from the other components of the sample. Generally, a bioanalytical method must be validated before it is implemented for routine use. To be validated, the bioanalytical method should be accurate. Internal standards are often used to ensure reliability of the quantitation of the bioanalytical method. In some cases, a stable isotope labelled compound of the drug is used as an internal standard. For an overview of bioanalysis in drug discovery and development, see S. Pandey et al., Pharm. Methods, 2010, 1(1), 14-24.

Inaccuracies in bioanalysis typically arise on sample preparation. Generally, samples are altered prior to drug detection, for example by removing proteins and/or concentrating the sample to improve detection of the components within the sample. Altering the sample is likely to change the concentration of the drug within the sample. In addition, where HPLC-ESI is used to analyse a sample, inaccuracies in the concentration of drug detected may arise from variations in the amount of sample injected into the HPLC column and variations in the degree of ionization of the drug. To account for such inaccuracies, known concentrations of internal standards are often added to samples prior to their preparation. Signals subsequently detected for the drugs of unknown concentration within the samples may then be calibrated according to the strength of the signal detected for the internal standard of known concentration. Advantageously, the internal standard is an isotopically-labelled version of the drug that has a similar extraction recovery, ionization response, and chromatographic retention time, and has a molecular weight that differs from the drug by a large enough number of m/z units to allow for separation of the signals arising from the internal standard and the drug (ideally 3 m/z units). (see N. R. Reddy, Mod. Appl. Pharm. Pharmacol., 2017, 1(2), 1-4; and J. Wu et al., J. Chromatogr. B Analyt Technol. Biomed. Life Sci., 2013, 941, 100-108).

The use of N,N-(dimethyl-d₆)-tryptamine (d₆-DMT) as an internal standard in the bioanalysis of plasma samples of DMT is described by G. N. Rossi et al., J. Pschedelic Stud., 2019, 3(1), 1-6; G. de Oliveira Silveria et al., Molecules, 2020, 25, 2072, 1-11; and C. D. R. Oliveira et al., Bioanalysis, 2012, 4(14), 1731-1738). To be particularly suitable as an internal standard, d₆-DMT should have a similar extraction recovery as DMT. However, since the six deuterium atoms of d₆-DMT are positioned just two bonds away from the amine, which is reported to undergo oxidation or cleavage on metabolism, the rate of metabolism of d₆-DMT in plasma is expected to be lower than that of DMT. Consequently, one would expect bioanalysis of DMT using d₆-DMT as an internal standard to be inaccurate.

There is a need in the art for internal standards for the bioanalysis of DMT-type compounds that are able to provide accurate quantitation. The present invention addresses this need.

### SUMMARY

The present invention relates to compositions comprising compounds of formula I and II, wherein ^{x}H, n, and R¹ are as defined below. As described above, there remains a need in the art for compounds with improved bioavailability and extended and/or modified pharmacokinetics and/or modified pharmacodynamics.

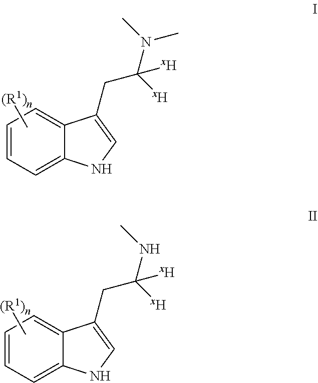

The inventors have demonstrated that increasing deuterium enrichment at the α-carbon of N,N-dimethyltryptamine increases metabolic stability, leading to a decrease in clearance and longer half-life. A linear relationship exists between deuterium enrichment at the α-carbon and half-life, in particular when the input reducing agent for production of the deuterium-enriched N,N-dimethyltryptamine-containing compositions of this invention comprise LiAlH₄ and LiAlD₄ with ratio between about 1:2.5 and about 2.5:1.

The binding of dimethylamino compounds of formula I to serotonin receptors within the body is expected to differ in selectivity and strength to the binding of monomethylamino compounds of formula II. Therefore, varying the relative amounts of the compounds of formula I and formula II within the compositions of the invention is expected to modulate the pharmacodynamics and consequently the therapeutic effect of the compositions. Combining compounds of formula I and formula II in varying amounts provides an additional variable through which the pharmacodynamics of the resultant compositions may be modified.

Accordingly, and viewed from a first aspect, the invention provides a composition comprising a compound of formula I and a compound of formula II:

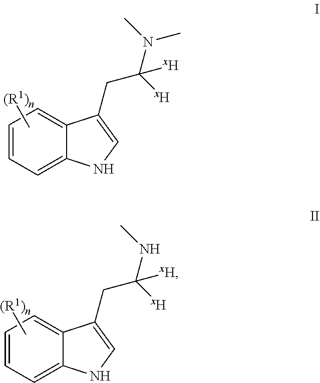

wherein:

each ^{x}H is independently selected from protium and deuterium;

n is selected from 0, 1, 2, 3 and 4;

each R¹ is independently selected from —R³, —OH, —OR³, —O(CO)R³, monohydrogen phosphate, —F, —Cl, —Br and —I; and

each R³ is independently selected from C₁-C₄alkyl.

Viewed from a second aspect, the invention provides a pharmaceutical composition comprising a composition of the first aspect, in combination with a pharmaceutically acceptable excipient.

The compositions of the first and second aspects have uses in the treatment of psychiatric or neurological disorders. Accordingly, viewed from a third aspect, the invention provides a composition of the first or second aspect for use in therapy.

Viewed from a fourth aspect, the invention provides a composition of the first or second aspect for use in a method of treating a psychiatric or neurological disorder in a patient.

Viewed from a fifth aspect, the invention provides a method of treatment comprising administering to a patient in need thereof a composition of the first or second aspect.

Viewed from a sixth aspect, the invention provides a chemical library comprising a plurality of compositions of the first or second aspect.

In order to assess the relative amounts of compounds of formula I and formula II within the compositions of the invention that provide desired pharmacokinetics and/or pharmacodynamics, it is useful to analyse the compositions using a bioanalytical technique. As described above, there is a need in the art for internal standards that are able to provide accurate quantitation in the bioanalysis of DMT-type compounds, such as those within the compositions of the invention. The inventors have found that compounds of formula III are useful internal standards for quantifying the amount of a target compound in a sample, such as an analogous compound comprising protio-monomethylamino (—N(H)CH₃) or protio-dimethylamino (—NCH₃)₂) groups.

Thus, the invention also relates to compounds of formula III, wherein ^{x}H, n, and R¹ are as defined below. In view of the kinetic isotope effect, it is expected that the deuterium atoms of the deutero-monomethylamino or deutero-dimethylamino groups of formula III would reduce the rate of metabolism of the compounds of formula III in plasma with respect to the rate of metabolism of target compounds comprising protio-monomethylamino or protio-dimethylamino groups. However, the inventors have found that the intrinsic clearance of compounds of formula III and their N,N-(dimethyl) or N-(monomethyl) analogues are surprisingly similar, indicating that the rate of metabolism of compounds of formula III and their N,N-(dimethyl) or N-(monomethyl) analogues in plasma is also similar. The inventors have found that compounds of formula III are useful internal standards, particularly for the quantitative measurement of analogous compounds comprising protio-N,N-dimethyl or protio-N,N-monomethyl groups.

Accordingly, viewed from a seventh aspect, the invention provides for the use of a compound of formula III:

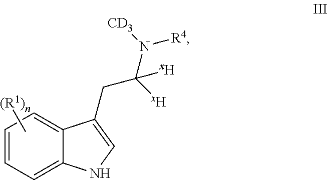

wherein:

each ^{x}H is independently selected from protium and deuterium;

n is selected from 0, 1, 2, 3 and 4;

each R¹ is independently selected from —R³, —OH, —OR³, —O(CO)R³, monohydrogen phosphate, —F, —Cl, —Br and —I; and

each R³ is independently selected from C₁-C₄ alkyl;

R⁴ is protium or —CD₃,

wherein n is 1, 2, 3 or 4 when each ^{x}H is protium and R⁴ is —CD₃, as an internal standard in an assay for quantifying the amount of a target compound in a sample.

Viewed from an eighth aspect, the invention provides a method of quantifying the amount of a target compound in a sample, the method comprising adding a known amount of a compound of formula III, as defined in the seventh aspect, to the sample.

Viewed from a ninth aspect, the invention provides a compound as defined in the seventh aspect, wherein n is 1, 2, 3 or 4 when each ^{x}H is protium.

Further aspects and embodiments of the present invention will be evident from the discussion that follows below.

### BRIEF DESCRIPTION OF THE FIGURES

FIG. 1: in vitro intrinsic clearance for DMT and 6 deuterium-containing compositions, as described in the Examples section, below. A) Linear regression analysis. The r² value for intrinsic clearance as a function of percentage of d₂-deuteration is 0.7485, p=0.01. B) Intrinsic clearance of deuterated analogues as a percent change from (undeuterated) DMT (dashed line).

FIG. 2: in vitro intrinsic clearance for DMT and 6 deuterium-containing compositions, as described in the Examples section, below. A) Linear regression analysis. The r² value for intrinsic clearance is 0.7648; where the slope was found to be significantly different to zero, p=0.01. B) Intrinsic clearance of deuterated analogues as a percent change from (undeuterated) DMT (dashed line).

FIG. 3: half-life for DMT and 6 deuterium-containing compositions, as described in the Examples section, below. A) Linear regression analysis. The r² value for half-life is 0.754; where the slope was found to be significantly different to zero, p=0.01. B) Half-life of deuterated analogues as a percent change from (undeuterated) DMT (dashed line).

FIG. 4: in vitro intrinsic clearance (A) and half-life (B) for d₂-deuterated and d₆-deuterated DMT blends, as described in the Examples section, below.

FIG. 5: in vitro intrinsic clearance (A) and half-life (B) for d₂-deuterated, d₆-deuterated and d₈-deuterated DMT blends, as described in the Examples section, below.

### DETAILED DESCRIPTION

Throughout this specification, one or more aspects of the invention may be combined with one or more features described in the specification to define distinct embodiments of the invention.

In the discussion that follows, reference is made to a number of terms, which are to be understood to have the meanings provided below, unless a context expressly indicates to the contrary. The nomenclature used herein for defining compounds, in particular the compounds described herein, is intended to be in accordance with the rules of the International Union of Pure and Applied Chemistry (IUPAC) for chemical compounds, specifically the “IUPAC Compendium of Chemical Terminology (Gold Book)” (see A. D. Jenkins et al., Pure & Appl. Chem., 1996, 68, 2287-2311). For the avoidance of doubt, if a rule of the IUPAC organisation is contrary to a definition provided herein, the definition herein is to prevail.

References herein to a singular of a noun encompass the plural of the noun, and vice-versa, unless the context implies otherwise. For example, “a compound of formula I” refers to one or more compounds of formula I.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps. The term “comprising” includes within its ambit the term “consisting”.

The term “consisting” or variants thereof is to be understood to imply the inclusion of a stated element, integer or step, or group of elements, integers or steps, and the exclusion of any other element, integer or step or group of elements, integers or steps.

The term “alkyl” is well known in the art and defines univalent groups derived from alkanes by removal of a hydrogen atom from any carbon atom, wherein the term “alkane” is intended to define acyclic branched or unbranched hydrocarbons having the general formula CₙH₂ₙ₊₂, wherein n is an integer C₁-C₄alkyl refers to any one selected from the group consisting of methyl, ethyl, n-propyl, iso-propyl, n-butyl, sec-butyl, iso-butyl and tert-butyl.

The term “acetoxy” (often abbreviated to OAc) defines a univalent group derived from acetic acid by removal of a hydrogen atom from the OH moiety. The term “methoxy” (often abbreviated to OMe) defines a univalent group derived from methanol by removal of a hydrogen atom from the OH moiety. The term monohydrogen phosphate defines a divalent group of formula HPO₄, derived from phosphoric acid by removal of a proton from two of the three OH moieties, and thus denotes a substituent of formula —OP(O)(OH)O⁻.

Where a compound of formula I or formula II is substituted with monohydrogen phosphate (i.e. where R¹ is monohydrogen phosphate), it is understood that “monohydrogen phosphate” also encompasses protonated or unprotonated analogues, i.e. dihydrogen phosphate and phosphate are also included. This is to reflect that psilocybin (also known as [3-(2-Dimethylaminoethyl)-1H-indol-4-yl] dihydrogen phosphate), and analogues such as [3-(2-methylaminoethyl)-1H-indol-4-yl] dihydrogen phosphate, in water generally comprise monohydrogen phosphate, this generally being understood to be the predominant form owing to the pKa values of the two terminal phosphate oxygen atoms being estimated as 1.3 and 6.5. It is further understood that the monohydrogen phosphate-containing form of psilocybin and analogues exists as a zwitterion (i.e. an internal salt) in which the nitrogen atom of the dimethylamino (or monomethylamino) moiety is protonated. For the avoidance of doubt, zwitterions are considered separately to salts, i.e. the pharmaceutically acceptable salts of the invention refer to salts comprising compounds of formula I or formula II of the invention and an acid. For example, a salt may be of psilocybin and fumaric acid.

The term “about” herein, when qualifying a number or value, is used to refer to values that lie within ±5% of the value specified. For example, if a percentage by weight of compound of formula I is specified to be about 5% to about 95%, percentages of 4.75% to 99.75% are included.

The compositions of the first and second aspects are useful in therapy and may be administered to a patient in need thereof. As used herein, the term ‘patient’ preferably refers to a mammal. Typically, the mammal is a human, but may also refer to a domestic mammal. The term does not encompass laboratory mammals.

The terms “treatment” and “therapy” define the therapeutic treatment of a patient, in order to reduce or halt the rate of progression of a disorder, or to ameliorate or cure the disorder. Prophylaxis of a disorder as a result of treatment or therapy is also included. References to prophylaxis are intended herein not to require complete prevention of a disorder: its development may instead be hindered through treatment or therapy in accordance with the invention. Typically, treatment or therapy is not prophylactic, and the compounds or compositions are administered to a patient having a diagnosed or suspected disorder.

Psychedelic-assisted psychotherapy means the treatment of a mental disorder by psychological means, which are enhanced by one or more protocols in which a patient is subjected to a psychedelic experience. A psychedelic experience is characterized by the striking perception of aspects of one's mind previously unknown, and may include one or more changes of perception with respect to hallucinations, synesthesia, altered states of awareness or focused consciousness, variation in thought patterns, trance or hypnotic states, and mystical states.

As is understood in the art, psychocognitive, psychiatric or neurological disorders are disorders which may be associated with one or more cognitive impairment. As used herein, the term ‘psychiatric disorder’ is a clinically significant behavioural or psychological syndrome or pattern that occurs in an individual and that is associated with present distress (e.g., a painful symptom) or disability (i.e., impairment in one or more important areas of functioning) or with a significantly increased risk of suffering death, pain, disability, or an important loss of freedom.

Diagnostic criteria for psychiatric or neurological disorders referred to herein are provided in the Diagnostic and Statistical Manual of Mental Disorders, Fifth Edition, (DSM-5).

As used herein the term ‘obsessive-compulsive disorder’ (OCD) is defined by the presence of either obsessions or compulsions, but commonly both. The symptoms can cause significant functional impairment and/or distress. An obsession is defined as an unwanted intrusive thought, image or urge that repeatedly enters the person's mind. Compulsions are repetitive behaviours or mental acts that the person feels driven to perform. Typically, OCD manifests as one or more obsessions, which drive adoption of a compulsion. For example, an obsession with germs may drive a compulsion to clean or an obsession with food may drive a compulsion to overeat, eat too little or throw up after eating (i.e. an obsession with food may manifest itself as an eating disorder). A compulsion can either be overt and observable by others, such as checking that a door is locked, or a covert mental act that cannot be observed, such as repeating a certain phrase in one's mind.

The term “eating disorder” includes anorexia nervosa, bulimia and binge eating disorder (BED). The symptoms of anorexia nervosa include eating too little and/or exercising too much in order to keep weight as low as possible. The symptoms of bulimia include eating a lot of food in a very short amount of time (i.e. binging) and then being deliberately sick, using laxatives, eating too little and/or exercising too much to prevent weight gain. The symptoms of BED include regularly eating large portions of food until uncomfortably full, and consequently feeling upset or guilty.

As used herein the term ‘depressive disorder’ includes major depressive disorder, persistent depressive disorder, bipolar disorder, bipolar depression, and depression in terminally ill patients.

As used herein the term ‘major depressive disorder’ (MDD, also referred to as major depression or clinical depression) is defined as the presence of five or more of the following symptoms over a period of two-weeks or more (also referred to herein as a ‘major depressive episode’), most of the day, nearly every day:
depressed mood, such as feeling sad, empty or tearful (in children and teens, depressed mood can appear as constant irritability);
significantly reduced interest or feeling no pleasure in all or most activities;
significant weight loss when not dieting, weight gain, or decrease or increase in appetite (in children, failure to gain weight as expected);
insomnia or increased desire to sleep;
either restlessness or slowed behaviour that can be observed by others;
fatigue or loss of energy;
feelings of worthlessness, or excessive or inappropriate guilt;
trouble making decisions, or trouble thinking or concentrating;
recurrent thoughts of death or suicide, or a suicide attempt.

At least one of the symptoms must be either a depressed mood or a loss of interest or pleasure.

Persistent depressive disorder, also known as dysthymia, is defined as a patient exhibiting the following two features:

A. has depressed mood for most the time almost every day for at least two years. Children and adolescents may have irritable mood, and the time frame is at least one year.

B. While depressed, a person experiences at least two of the following symptoms:
Either overeating or lack of appetite.
Sleeping too much or having difficulty sleeping.
Fatigue, lack of energy.
Poor self-esteem.
Difficulty with concentration or decision-making.

As used herein the term ‘treatment resistant major depressive disorder’ describes MDD that fails to achieve an adequate response to an adequate treatment with standard of care therapy.

As used herein, ‘bipolar disorder’, also known as manic-depressive illness, is a disorder that causes unusual shifts in mood, energy, activity levels, and the ability to carry out day-to-day tasks.

There are two defined sub-categories of bipolar disorder; all of them involve clear changes in mood, energy, and activity levels. These moods range from periods of extremely “up,” elated, and energised behaviour (known as manic episodes, and defined further below) to very sad, “down,” or hopeless periods (known as depressive episodes). Less severe manic periods are known as hypomanic episodes.

Bipolar I Disorder—defined by manic episodes that last at least 7 days, or by manic symptoms that are so severe that the person needs immediate hospital care. Usually, depressive episodes occur as well, typically lasting at least 2 weeks. Episodes of depression with mixed features (having depression and manic symptoms at the same time) are also possible.

Bipolar II Disorder—defined by a pattern of depressive episodes and hypomanic episodes, but not the full-blown manic episodes described above.

As used herein ‘bipolar depression’ is defined as an individual who is experiencing depressive symptoms with a previous or coexisting episode of manic symptoms, but does not fit the clinical criteria for bipolar disorder.

As used herein, the term ‘anxiety disorder’ includes generalised anxiety disorder, phobia, panic disorder, social anxiety disorder, and post-traumatic stress disorder.

‘Generalised anxiety disorder’ (GAD) as used herein means a chronic disorder characterised by long-lasting anxiety that is not focused on any one object or situation. Those suffering from GAD experience non-specific persistent fear and worry, and become overly concerned with everyday matters. GAD is characterised by chronic excessive worry accompanied by three or more of the following symptoms: restlessness, fatigue, concentration problems, irritability, muscle tension, and sleep disturbance.

‘Phobia’ is defined as a persistent fear of an object or situation the affected person will go to great lengths to avoid, typically disproportional to the actual danger posed. If the feared object or situation cannot be avoided entirely, the affected person will endure it with marked distress and significant interference in social or occupational activities.

A patient suffering from a ‘panic disorder’ is defined as one who experiences one or more brief attack (also referred to as a panic attack) of intense terror and apprehension, often marked by trembling, shaking, confusion, dizziness, nausea, and/or difficulty breathing. A panic attack is defined as a fear or discomfort that abruptly arises and peaks in less than ten minutes.

‘Social anxiety disorder’ is defined as an intense fear and avoidance of negative public scrutiny, public embarrassment, humiliation, or social interaction. Social anxiety often manifests specific physical symptoms, including blushing, sweating, and difficulty speaking.

‘Post-traumatic stress disorder’ (PTSD) is an anxiety disorder that results from a traumatic experience. Post-traumatic stress can result from an extreme situation, such as combat, natural disaster, rape, hostage situations, child abuse, bullying, or even a serious accident. Common symptoms include hypervigilance, flashbacks, avoidant behaviours, anxiety, anger and depression.

As used herein, the term “post-partum depression” (PPD, also known as postnatal depression) is a form of depression experienced by either parent of a newborn baby. Symptoms typically develop within 4 weeks of delivery of the baby and often include extreme sadness, fatigue, anxiety, loss of interest or pleasure in hobbies and activities, irritability, and changes in sleeping or eating patterns.

As used herein, the term ‘substance abuse’ means a patterned use of a drug in which the user consumes the substance in amounts or with methods that are harmful to themselves or others.

As used herein, the term ‘an avolition disorder’ refers to a disorder that includes as a symptom the decrease in motivation to initiate and perform self-directed purposeful activities.

As described above, the invention provides in its first aspect a composition comprising a compound of formula I and a compound of formula II:

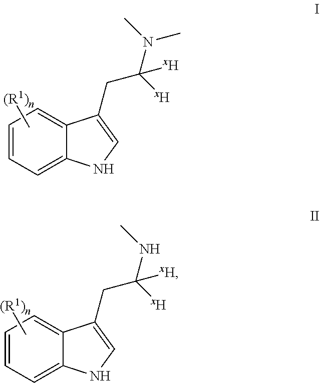

wherein:

each ^{x}H is independently selected from protium and deuterium;

n is selected from 0, 1, 2, 3 and 4;

each R¹ is independently selected from —R³, —OH, —OR³, —O(CO)R³, monohydrogen phosphate, —F, —Cl, —Br and —I; and

each R³ is independently selected from C₁-C₄alkyl.

Varying the relative amounts of the compounds of formula I and formula II within the compositions of the invention is expected to modulate the pharmacodynamics and consequently the therapeutic effect of the compositions. Moreover, greater concentrations of monomethyltryptamine compounds of formula II are tolerated in a subject than the dimethyltryptamine analogue of formula I. The ratio of the compound of formula I:the compound of formula II within the composition may be varied depending on the metabolic profile of the patient to whom the composition is intended to be administered—greater ratios of compounds of formula II may be more suitable for a patient with a higher metabolism.

In some embodiments, the composition comprises about 5% to about 95% by weight of the compound of formula I or about 95% to about 5% by weight of the compound of formula II.

In some embodiments, the composition comprises about 10% to about 90%, about 20% to about 80%, about 30% to about 70%, about 40% to about 60% or about 50% by weight of the compound of formula I. In some embodiments, the composition comprises about 90% to about 10%, about 80% to about 20%, about 70% to about 30%, about 60% to about 40% or about 50% by weight of the compound of formula II.

In some embodiments, the composition comprises a ratio of compound of formula I:compound of formula II by weight of 20:1, 15:1, 10:1, 8:1, 6:1, 4:1, 2:1, 1:1, 0.8:1, 0.6:1. 0.4:1, 0.2:1, 0.1:1 or 0.05:1.

For the avoidance of doubt, where the composition comprises two or more compounds of formula I or II, the above-mentioned percentage weights and ratios refer to the combined weight of each compound of formula I or each compound of formula II.

Each ^{x}H is independently selected from protium and deuterium, wherein deuterium is a hydrogen atom with an additional neutron. For the avoidance of doubt, by ^{x}H being deuterium is meant that the compounds are enriched with deuterium, i.e. they comprise a percentage of deuterium that is greater than the natural abundance of deuterium, which is about 0.015%. In some embodiments, the compounds of formula I or II that are substituted with deuterium are enriched with deuterium by an amount that is dependent on the percentage of deuterium available in the reagents from which the compounds are derived. For example, and as described below, the d₆-dimethylamino or d₃-monomethylamino substituents of formula III may be derived from dimethyl-d₆-amine or methyl-d₃-amine (commonly available as HCl salts), which are available from chemical vendors in purities of deuterium that range from 98% to 99%. The purity of deuterium in the resultant d₆-dimethylamino or d₃-monomethylamino substituents is consequently 98% to 99%. This means that not all compounds of formula III comprise d₆-dimethylamino or d₃-monomethylamino substituents—some may comprise do-d₅dimethylamino or do-d₃-monomethylamino substituents, but the average purity of deuterium is about 98% to 99%.

In some embodiments, the composition comprises a compound of formula I and a compound of formula II in both of which one ^{x}H is H and the other is D. In some embodiments, the composition comprises a compound of formula I and a compound of formula II in both of which each ^{x}H is H. Sometimes, the composition comprises a compound of formula I and a compound of formula II in both of which each ^{x}H is D.

Often, the composition of the first aspect comprises a compound of formula I and a compound of formula II in both of which at least one ^{x}H is D. The inventors have found that such compounds of formula I or II comprising at least one deuterium atom at the α-position are metabolised substantially more slowly than their α-diprotic analogues and consequently exhibit long lasting therapeutic effects.

For the avoidance of doubt, the above-mentioned embodiments do not exclude the presence of further compounds of formula I or formula II. For example, the composition may comprise a compound of formula I and a compound of formula II in both of which one ^{x}H is H and the other is D, a compound of formula I and a compound of formula II in both of which each ^{x}H is H and a compound of formula I and a compound of formula II in both of which each ^{x}H is D.

As described above, each R¹ is independently selected from —R³, —OH, —OR³, —O(CO)R³, monohydrogen phosphate, —F, —Cl, —Br and —I, and each R³ is independently selected from C₁-C₄alkyl. In some embodiments, each R¹ is independently selected from —OH, —OR³, —OC(O)R³ and monohydrogen phosphate.

Sometimes, each R³ is independently selected from methyl or ethyl. In some embodiments, each R³ is independently selected from methyl. Often, each R¹ is independently selected from —OH, —OMe, —OC(O)Me and monohydrogen phosphate.

n is selected from 0, 1, 2, 3 and 4. In some embodiments, n is 0 or 1. When n is 1, R¹ is typically bound to the 4- or 5-position. For the avoidance of doubt, positions 4 and 5 refer to the positions labelled in the structures below (substitution not shown).

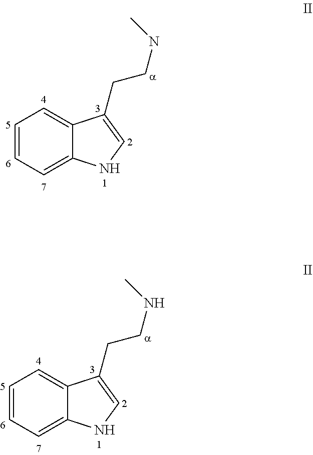

In some embodiments, n is 0, or n is 1 and R¹ is selected from 5-methoxy, 4-acetoxy, 4-monohydrogen phosphate, 4-hydroxy and 5-hydroxy, such as 5-methoxy, 4-acetoxy, 4-monohydrogen phosphate and 4-hydroxy. Sometimes, n is 1 and R¹ is selected from 5-methoxy, 4-acetoxy, 4-monohydrogen phosphate and 4-hydroxy. In some embodiments, n is 0, or n is 1 and R¹ is 5-methoxy.

Sometimes, the composition comprises a compound of formula I selected from the group consisting of N,N-dimethyltryptamine, α-monodeutero-N,N-dimethyltryptamine, α,α-dideutero-N,N-dimethyltryptamine, 5-methoxy-N,N-dimethyltryptamine, 5-methoxy-α-monodeutero-N,N-dimethyltryptamine, and 5-methoxy-α,α-dideutero-N,N-dimethyltryptamine.

Sometimes, the composition comprises a compound of formula II selected from the group consisting of N-monomethyltryptamine, α-monodeutero-N-monomethyltryptamine, α,α-dideutero-N-monomethyltryptamine, 5-methoxy-N-monomethyltryptamine, 5-methoxy-α-monodeutero-N-monomethyltryptamine, and 5-methoxy-α,α-dideutero-N-monomethyltryptamine.

In some embodiments, the composition comprises a compound of formula I and a compound of formula II in both of which ^{x}H, n, and R¹ are the same. For example, the composition may comprise N,N-dimethyltryptamine and N-monomethyltryptamine, α-monodeutero-N,N-dimethyltryptamine and α-monodeutero-N-monomethyltryptamine, α,α-dideutero-N,N-dimethyltryptamine and α,α-dideutero-N-monomethyltryptamine, 5-methoxy-N,N-dimethyltryptamine and 5-methoxy-N-monomethyltryptamine, 5-methoxy-α-monodeutero-N,N-dimethyltryptamine and 5-methoxy-α-monodeutero-N-monomethyltryptamine, or 5-methoxy-α,α-dideutero-N,N-dimethyltryptamine and 5-methoxy-α,α-dideutero-N-monomethyltryptamine.

In some embodiments, the composition comprises two compounds of formula I, which differ from one another only by the definition of ^{x}H. In particular embodiments, one of the two compounds of formula I is an undeuterated analogue of the other, for example one may be α-monodeutero-N,N-dimethyltryptamine or α,α-dideutero-N-monomethyltryptamine and the other may be N,N-dimethyltryptamine.

In some embodiments, the composition comprises two compounds of formula II, which differ from one another only by the definition of ^{x}H. In particular embodiments, one of the two compounds of formula II is an undeuterated analogue of the other.

Sometimes, the composition comprises two compounds of formula I, which differ from one another only by the definition of ^{x}H and two compounds of formula II, which differ from each other only by the definition of ^{x}H. n, and R¹ of the compounds of formula I and formula II may be the same. For example, the composition may comprise α-monodeutero-N,N-dimethyltryptamine, N,N-dimethyltryptamine, α-monodeutero-N-monomethyltryptamine and N-monomethyltryptamine.

Sometimes, the composition comprises three compounds of formula I, which differ from one another only by the definition of ^{x}H. Sometimes, the composition comprises three compounds of formula II, which differ from one another only by the definition of ^{x}H. In these embodiments, the composition comprises the undeuterated, monodeuterated and dideuterated analogues of a compound of formula I or formula II.

Sometimes, the composition comprises three compounds of formula I, which differ from one another only by the definition of ^{x}H and three compounds of formula II, which differ from each other only by the definition of ^{x}H. n, and R¹ of the compounds of formula I and formula II may be the same. For example, the composition may comprise α,α-dideutero-N,N-dimethyltryptamine, α-monodeutero-N,N-dimethyltryptamine, N,N-dimethyltryptamine, α,α-dideutero-N-monomethyltryptamine, α-monodeutero-N-monomethyltryptamine and N-monomethyltryptamine.

The mean molecular weight ranges of particular compounds of formula I (mixtures of protio and deutero analogues thereof) and compounds of formula II (mixtures of protio and deutero analogues thereof) are shown in Table 1.

TABLE 1
Mean molecular weight ranges of preferred compounds

| | | Preferred m/w range of |
|---|---|---|
| | | compounds of formula I or |
| Compound | R¹ | formula II in Daltons |
| DMT | — | 188.4-190.3 |
| 4-OAc-DMT | 4-OC(O)CH₃ | 246.4-248.3 |
| 5-OAc-DMT | 5-OC(O)CH₃ | 246.4-248.3 |
| 5-MeO-DMT | 5-OCH₃ | 218.4-220.3 |
| NMT | — | 174.4-176.3 |
| 4-OAc-NMT | 4-OC(O)CH₃ | 232.4-234.3 |
| 5-OAc-NMT | 5-OC(O)CH₃ | 232.4-234.3 |
| 5-MeO-NMT | 5-OCH₃ | 204.4-206.3 |

Particular compositions of the invention may be formed by combining the compounds of formula I and II as defined in Table 1, e.g. by combining DMT with a mean molecular weight range of 188.4-190.3 gmol⁻¹ with NMT with a mean molecular weight range of 174.4-176.3 gmol⁻¹.

As used herein, mean molecular weight means the weighted average of molecular weights of the compounds of formula I or formula II, as measured by an appropriate mass spectroscopic technique, for example LC-MS SIM (selected-ion monitoring). In some embodiments, the mean molecular weight is the weighted average.

It will be understood that providing compositions with such specific mean molecular weights can be achieved by those skilled in the art through the teachings herein, in particular by adjusting the relative proportions of lithium aluminium hydride and lithium aluminium deuteride in the reductions exemplified.

For the avoidance of doubt, when the composition comprises more than one compound of formula I or formula II and at least one ^{x}H of at least one of the compounds within the composition is deuterium, that ^{x}H is deuterated by a greater amount than found in isotopically unenriched protio analogues. It will also be understood that the greater the proportion of compounds in which at least one ^{x}H is deuterium, the higher the mean molecular weight of the composition.

In some embodiments, the composition consists essentially of a compound of formula I and a compound of formula II. This means that the composition does not comprise material quantities of other pharmaceutically active compounds, including other dimethyltryptamine compounds.

In other words, and alternatively put, the compositions according to these specific embodiments constitute a drug substance comprising a biologically active ingredient consisting essentially of a mixture of a compound of formula I and a compound of formula II.

Deuterated DMT or NMT compounds can be synthesised following the reaction scheme (synthetic schemes) provided in Scheme 1 below. The chemistry depicted in this scheme was reported by PE Morris and C Chiao (Journal of Labelled Compounds And Radiopharmaceuticals, Vol. XXXIII, No. 6, 455-465 (1993)). Deuterated DMT or NMT compounds can also be synthesised following the synthetic scheme depicted in Scheme 2.

Compositions may comprise specific amounts of N,N-dimethyltryptamines and N-methyltryptamines (for example DMT or NMT itself or R⁴- or R⁵-substituted DMTs or NMTs described herein) and/or deuterated N,N-dimethyltryptamine and N-methyltryptamine compounds, with the relative proportions of the protio against the deutero compounds controlled by varying the ratio of lithium aluminium hydride and lithium aluminium deuteride in the reducing agent. Relative proportions may further be varied by adding one or more of the protio or deutero compounds to the compositions described herein.

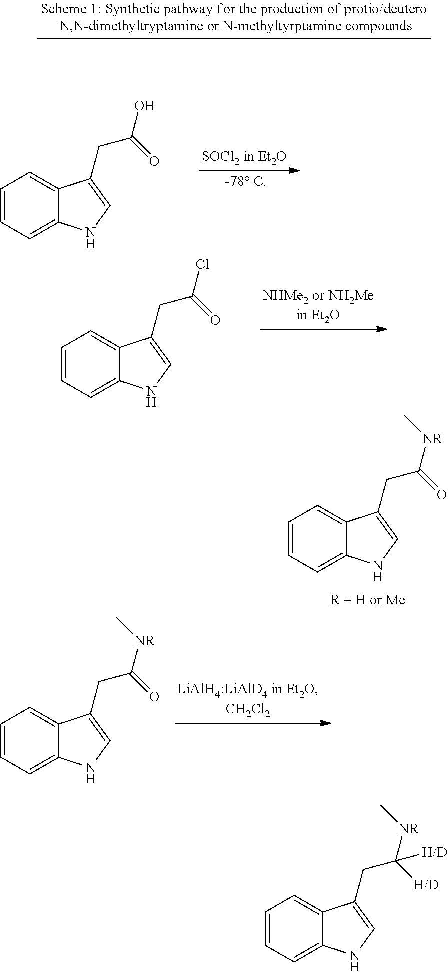

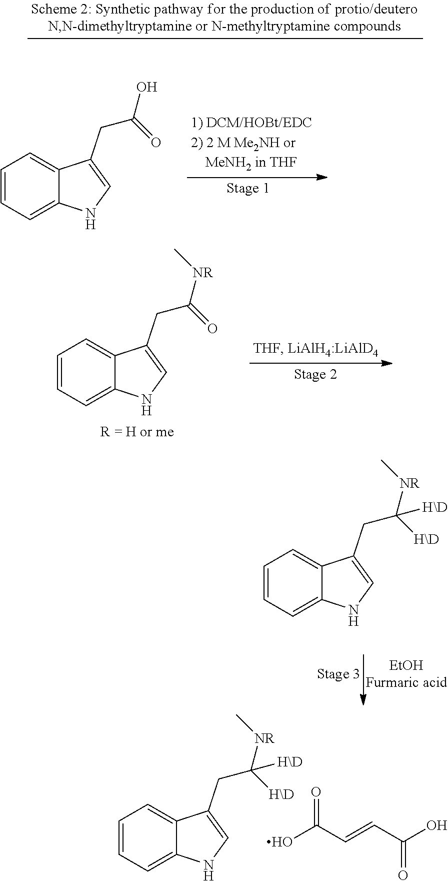

Identification of the compositions resultant from the reduction step in Schemes 1 and 2 may be achieved, if desired, by chromatographic separation of the components of the mixtures by conventional means at the disposal of the skilled person in combination with spectroscopic and/or mass spectrometric analysis.

Alternative compositions are obtainable by mixing protio compounds, obtainable by Scheme 1 or Scheme 2 when the reducing agent is exclusively lithium aluminium hydride, with an α,α-dideutero compound obtainable from Scheme 1 or Scheme 2 when the reducing agent is exclusively lithium aluminium deuteride.

The compositions described hereinabove may be further modified by adding one or more α-monodeutero compounds. Stocks of such compounds may be obtained, for example, from the chromatographic separation described above.

Scheme 3 represents schemes known in the art to synthesise DMT or NMT compounds, in which substituent R¹ denotes hydrogen or the substituent R¹ as defined in formulae I and II, and R² is methyl or H.

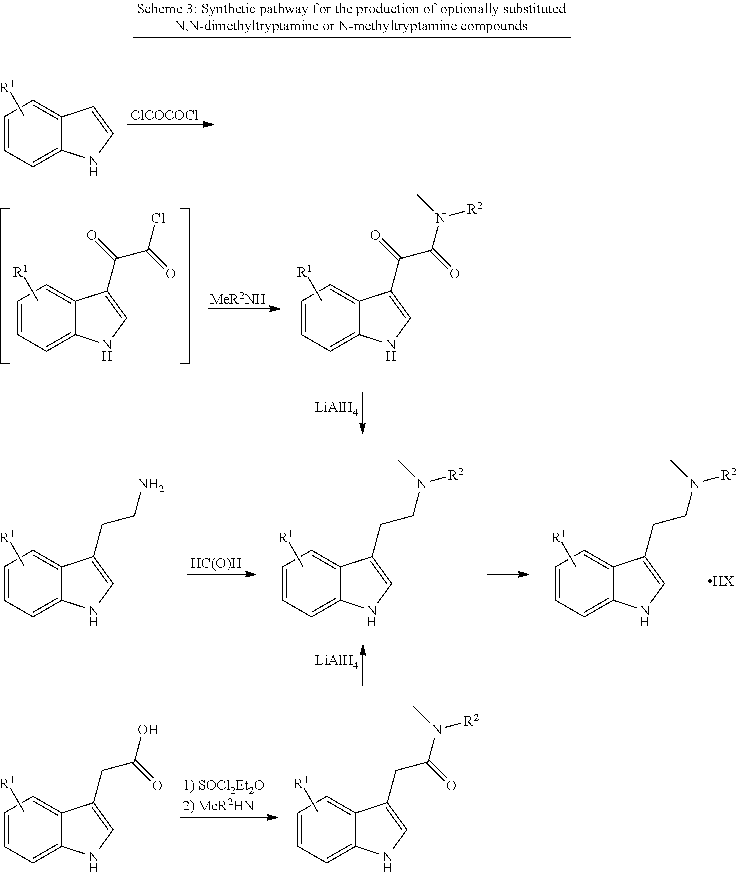

Mixtures of compounds comprising controllable proportions of DMT or NMT and the same DMT or NMT but with α-mono- and/or α,α-di-deuteration may, if desired, be prepared by reducing 2-(3-indolyl)-N,N-dimethyl acetamide or 2-(3-indolyl)-N-methyl acetamide with a desired ratio of lithium aluminium hydride and lithium aluminium deuteride. Analogous mixtures of compounds comprising controllable proportions of R¹-substituted DMT or NMT may be prepared by reducing R¹-substituted 2-(3-indolyl)-N,N-dimethyl acetamide, R¹-substituted 2-(3-indolyl)-N-methyl acetamide, R¹-substituted 2-(3-indolyl)-N,N-dimethyl oxoacetamide or R¹-substituted 2-(3-indolyl)-N-methyl oxoacetamide with a desired ratio of lithium aluminium hydride and lithium aluminium deuteride.

In some cases, protecting groups may be useful. For example, to synthesise DMT or NMT compounds substituted with hydroxy, monohydrogen phosphate or acetyl, a benzyloxy 2-(3-indolyl)-N,N-dimethyl oxoacetamide may be reduced with a desired ratio of lithium aluminium hydride and lithium aluminium deuteride to produce a benzyloxy-N,N-dimethyl tryptamine (optionally substituted at the α and/or β positions with deuterium). The benzyl protecting group may then be removed, e.g. by hydrogenating with hydrogen and palladium on carbon, to form a hydroxy-N,N-dimethyl tryptamine (optionally substituted at the α and/or β positions with deuterium). The hydroxy group may be converted to a monohydrogen phosphate or an acetyl by reaction with either tetra-O-benzyl-pyrophosphate (followed by removal of the benzyl protecting group) or reaction with acetic anhydride. See D. E. Nichols and S. Frescas, Synthesis, 1999, 6, 935-938 for further information on this synthetic strategy.

Methods by which the compounds of formula I or formula II may be produced are suitable for the production of high purity compounds of formula I or formula II. The composition may comprise a drug substance, which comprises a compound of formula I and a compound of formula II, wherein each compound is at a purity of greater than or equal to 99% when measured by HPLC. In some embodiments, each compound of formula I and II is of a purity of greater than or equal to 99% by HPLC. Often, each compound of formula I and II is of a purity of between 99% and 100% by HPLC, such as a purity of between 99.5% and 100% by HPLC. In some embodiments, each compound of formula I and II, is of a purity of between 99.9% and 100% by HPLC, such as a purity of between 99.95% and 100% by HPLC.

In some embodiments, the composition has an oxygen content of less than 2 ppm, such as between 0.1 ppm and 2 ppm. The skilled person is able to determine the oxygen content of the formulation using any technique known in the art to be suitable, such as using a dissolved oxygen meter (e.g. a Jenway 970 Enterprise Dissolved Oxygen Meter, available from Keison Products: http://www.keison.co.uk/products/jenway/970.pdf.

The composition may be stored in any suitable container. In some embodiments, to ameliorate degradation of the composition, the composition is stored in a container adapted to prevent penetration of ultraviolet light, such as an amber glass vial. In others, the container within which the composition is stored is not so adapted (and may be, for example, made of clear glass) with protection against ultraviolet light, if desired, provided by secondary packaging (for example packaging within which the receptacle containing the formulation may be placed).

To ameliorate degradation of the composition, it may be desirable to minimise the total oxygen content within the container in which the composition is stored, the oxygen within the container equilibrating between the composition and the headspace (if any) within the container. Accordingly, it may be desirable to store the composition under an inert atmosphere for example by purging the headspace to reduce its oxygen content from about 20% typically found in air, to less than, for example, 0.5%. Often, the container is airtight and the composition is stored under an inert atmosphere, such as under nitrogen or argon, typically nitrogen. The composition may be stored at room temperature, e.g. at about 20 to about 30° C. or at cooler temperatures, for example at about 2 to about 8° C. Alternatively, to ameliorate degradation of the composition further, it may be stored at temperatures lower than room temperature, such as in a refrigerator or freezer.

In some embodiments, the compounds of formula I and formula II are in the form of pharmaceutically acceptable salts. The salts comprise an acid and the compound of formula I or formula II. An example of a salt comprising a compound of formula I is dimethyltryptamine fumarate, which is the fumaric acid salt of dimethyltryptamine. P. H. Stahl and C. G. Wermuth provide an overview of pharmaceutical salts and the acids comprised therein in Handbook of Pharmaceutical Salts: Properties, Selection and Use, Weinheim/Zürich:Wiley-VCHNHCA, 2002. The acids described in this review are suitable acids for inclusion within the pharmaceutically acceptable salts of the invention.

The salts may comprise an acid selected from the group consisting of fumaric acid, tartaric acid, citric acid, acetic acid, lactic acid, gluconic acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, adipic acid, ascorbic acid, aspartic acid, benzenesulfonic acid, benzoic acid, camphoric acid, camphor-10-sulfonic acid, decanoic acid, hexanoic acid, octanoic acid, carbonic acid, cinnamic acid, cyclamic acid, dodecylsulfuric acid, ethane-1,2-disulfonic acid, ethanesulfonic acid, formic acid, galactaric acid, gentisic acid, glucoheptonic acid, glucuronic acid, glutamic acid, glutaric acid, glycerophosphoric acid, glycolic acid, hippuric acid, hydrobromic acid, hydrochloric acid, isobutyric acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (-L), salicylic acid, sebacic acid, stearic acid, succinic acid, sulfuric acid, thiocyanic acid, toluenesulfonic acid and undecylenic acid.

In some embodiments, the salts comprise fumaric acid.

As described above, the invention provides in its second aspect a pharmaceutical composition comprising a composition of the first aspect in combination with a pharmaceutically acceptable excipient.

For the avoidance of doubt, embodiments related to the composition of the first aspect, as defined herein, apply mutatis mutandis to the second aspect. For example, the composition may comprise a compound of formula I and a compound of formula II in both of which one ^{x}H is H and the other is D; n may be 0, or n may be 1 and R¹ is 5-methoxy; and/or n, and R¹ of both compounds may be the same.

Examples of pharmaceutically acceptable excipients that may be comprised within the composition of the first aspect include but are not limited to those described in Gennaro et al., Remmington: The Science and Practice of Pharmacy, 20^{th} Edition, Lippincott, Williams and Wilkins, 2000 (specifically part 5: pharmaceutical manufacturing). Suitable pharmaceutically acceptable excipients are also described in the Handbook of Pharmaceutical Excipients, 2^{nd} Edition; Editors A. Wade and P. J. Weller, American Pharmaceutical Association, Washington, The Pharmaceutical Press, London, 1994. M. F. Powell, T. Nguyen and L. Baloian provide a review of excipients suitable for parenteral administration (administration other than by the mouth or alimentary canal) in PDA J. Pharm. Sci. Technol., 52, 238-311 (1998). Compositions include those suitable for oral, nasal, topical (including buccal, sublingual and transdermal), parenteral (including subcutaneous, intravenous and intramuscular) or rectal administration.

By means of pharmaceutically suitable liquids, the compositions of the invention can be prepared in the form of a solution, suspension, emulsion, or as a spray. Aqueous suspensions, isotonic saline solutions and sterile injectable solutions may be used, containing pharmaceutically acceptable dispersing agents and/or wetting agents, such as propylene glycol or butylene glycol.

The invention also provides a composition of the invention, in combination with packaging material suitable for the composition, the packaging material including instructions for the use of the composition.

The invention provides in its third aspect a composition of the first or second aspect for use in therapy.

For the avoidance of doubt, embodiments related to the composition of the first and second aspects, as defined herein, apply mutatis mutandis to the third aspect. For example, the composition may comprise a compound of formula I and a compound of formula II in both of which one ^{x}H is H and the other is D; n may be 0, or n may be 1 and R¹ is 5-methoxy; n, and R¹ of both compounds may be the same; and/or the composition may comprise a pharmaceutically acceptable excipient described in Gennaro et al., 2000 (supra).

In some embodiments, the therapy is psychedelic-assisted psychotherapy, i.e. the therapy is treatment of a mental disorder by psychological means, which are enhanced by one or more protocols in which a patient is subjected to a psychedelic experience induced by administration of the formulation.

The invention provides in its fourth aspect a composition of the first or second aspect for use in a method of treating a psychiatric or neurological disorder in a patient.

In another aspect, the invention provides use of a composition of the first or second aspect for the manufacture of a medicament. In some embodiments of this aspect, the medicament is for use in a method of treating a psychiatric or neurological disorder in a patient.

In some embodiments, the psychiatric or neurological disorder is selected from (i) an obsessive compulsive disorder, (ii) a depressive disorder, (iii) a schizophrenia disorder, (iv) a schizotypal disorder, (v) an anxiety disorder, (vi) substance abuse, and (vii) an avolition disorder. Often, the psychiatric or neurological disorder is selected from the group consisting of (i) an obsessive compulsive disorder, (ii) a depressive disorder, (iii) an anxiety disorder, (iv) substance abuse, and (v) an avolition disorder.

In some embodiments, the disorder is selected from the group consisting of major depressive disorder, treatment resistant major depressive disorder, post-partum depression, an obsessive compulsive disorder and an eating disorder such as a compulsive eating disorder.

In some embodiments, the psychiatric or neurological disorder is major depressive disorder. In some embodiments, the psychiatric or neurological disorder is treatment resistant depression.

The invention provides in its fifth aspect a method of treatment comprising administering to a patient in need thereof a composition of the first or second aspect.

In some embodiments, the method of treatment is psychedelic-assisted psychotherapy, i.e. the method of treatment is treatment of a mental disorder by psychological means, which are enhanced by one or more protocols in which a patient is subjected to a psychedelic experience induced by administration of the formulation.

For the avoidance of doubt, embodiments related to the method of treatment, of the fourth aspect of the invention apply mutatis mutandis to the fifth aspect. For example, the disorder may be selected from the group consisting of (i) an obsessive compulsive disorder, (ii) a depressive disorder, (iii) an anxiety disorder, (iv) substance abuse, and (v) an avolition disorder.

In order to treat the disorder, an effective amount of the composition is administered, i.e. an amount that is sufficient to reduce or halt the rate of progression of the disorder, or to ameliorate or cure the disorder and thus produce the desired therapeutic or inhibitory effect.

The invention provides in its sixth aspect a chemical library comprising a plurality of compositions of the first or second aspects.

For the avoidance of doubt, embodiments related to the composition of the first and second aspects, as defined herein, apply mutatis mutandis to the sixth aspect. For example, the composition may comprise a compound of formula I and a compound of formula II in both of which one ^{x}H is H and the other is D; n may be 0, or n may be 1 and R¹ is 5-methoxy; n, and R¹ of both compounds may be the same; and/or the composition may comprise a pharmaceutically acceptable excipient described in Gennaro et al., 2000 (supra).

The plurality of compositions within the library may be characterised by one or more parameters selected from mean molecular weight and mean alpha-deuterium saturation. By mean alpha-deuterium saturation is meant a measure of the average number of deuterium atoms at the alpha position of the compounds of formula I and formula II within the composition, as measured by an appropriate mass spectroscopic technique, for example LC-MS SIM. Since the number of deuterium atoms at the alpha position of compounds of formula I or formula II may be 0, 1 or 2, the mean alpha-deuterium saturation is a number ranging from 0 to 2.

As described above, in order to assess the relative amounts of compounds of formula I and formula II within the compositions of the invention that provide suitable pharmacokinetic and/or pharmacodynamic profiles, it is useful to analyse the compositions using a bioanalytical technique. Such a bioanalytical technique may be used to analyse the concentration of compounds of formula I and compounds of formula II within the plasma of a subject to whom one or more compositions of the invention have been administered. Analysing the decrease in concentration of compounds of formula I and compounds of formula II within the plasma of a subject over time allows the half-life of the compounds within the subject to be calculated. The compositions within the library may be characterised by the mean half-life (within a subject) of the compounds of formula I and II comprised within the compositions. By mean half-life is meant a measure of the average time required for the concentration of compounds of formula I and formula II to reduce to half of their initial concentration within a subject, the concentrations measured by an appropriate mass spectroscopic technique, for example LC-MS SIM.

As described above, there is a need in the art for internal standards for the bioanalysis of DMT-type compounds such as those within the compositions of the invention, that are able to provide accurate quantitation. The inventors have found that compounds of formula III are useful internal standards for quantifying the amount of a target compound in a sample, such as an analogous compound comprising protio-monomethylamino or protio-dimethylamino groups.

Accordingly, the invention provides in its seventh aspect a compound of formula III:

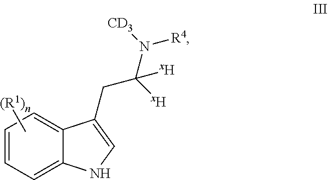

wherein:

each ^{x}H is independently selected from protium and deuterium;

n is selected from 0, 1, 2, 3 and 4;

each R¹ is independently selected from —R³, —OH, —OR³, —O(CO)R³, monohydrogen phosphate, —F, —Cl, —Br and —I; and

each R³ is independently selected from C₁-C₄ alkyl;

R⁴ is protium or —CD₃,
wherein n is 1, 2, 3 or 4 when each ^{x}H is protium and R⁴ is —CD₃, as an internal standard in an assay for quantifying the amount of a target compound in a sample.

In some embodiments, n is 1, 2, 3 or 4, in particular 1, when each ^{x}H is protium.

Sometimes, each R¹ is independently selected from —R³, —OH, —OR³, —OC(O)R³, monohydrogen phosphate, —F, —Cl, —Br and —I. In some embodiments, each R¹ is independently selected from —OH, —OR³, —OC(O)R³ and monohydrogen phosphate.

Sometimes, each R³ is independently selected from methyl or ethyl. In some embodiments, each R³ is independently selected from methyl. Often, each R¹ is independently selected from —OH, —OMe, —OC(O)Me and monohydrogen phosphate.

In some embodiments, n is 0 or 1. When n is 1, R¹ is typically bound to the 4- or 5-position. In some embodiments, n is 0, or n is 1 and R¹ is selected from 5-methoxy, 4-acetoxy, 4-monohydrogen phosphate, 4-hydroxy and 5-hydroxy, such as 5-methoxy, 4-acetoxy, 4-monohydrogen phosphate and 4-hydroxy. Sometimes, n is 1 and R¹ is selected from 5-methoxy, 4-acetoxy, 4-monohydrogen phosphate and 4-hydroxy. In some embodiments, n is 0, or n is 1 and R¹ is 5-methoxy.

Sometimes, the compound of formula III is any one selected from the group consisting of α-monodeutero-N,N-(dimethyl-d₆)tryptamine, α,α-dideutero-N,N-(dimethyl-d₆)tryptamine, 5-methoxy-N,N-(dimethyl-d₆)tryptamine, 5-methoxy-α-monodeutero-N,N-(dimethyl-d₆)tryptamine, 5-methoxy-α,α-dideutero-N,N-(dimethyl-d₆)tryptamine, N-(monomethyl-d₃)tryptamine, α-monodeutero-N-(monomethyl-d₃)tryptamine, α,α-dideutero-N-(monomethyl-d₃)tryptamine, 5-methoxy-N-(monomethyl-d₃)tryptamine, 5-methoxy-α-monodeutero-N-(monomethyl-d₃)tryptamine, and 5-methoxy-α,α-dideutero-N-(monomethyl-d₃)tryptamine.

By “target compound” is meant an analyte of interest, which is, typically a biologically active compound within a sample. The target compound need not be analogous to the internal standard. Oliveira et al., 2012 (supra) describe the use of d₆-DMT as an internal standard in an assay for quantifying the amount of DMT, harmine (HRM), harmaline (HRL) and tetrahydroharmine (THH) within plasma. The structures of HRM, HRL and THH differ from the structure of DMT, yet the authors report the use of d₆-DMT to quantify the amount of these compounds within plasma.

The internal standard is typically an isotopically labelled version of the target compound. It is advantageous that the internal standard has a similar extraction recovery, ionization response, and chromatographic retention time as the target compound, and has a molecular weight that differs from the target compound by a large enough number of m/z units to allow for separation of the signals arising from the internal standard and the target compound (ideally 3 m/z units). In some embodiments, the target compound has a mean molecular weight that differs from the internal standard by at least 2.5 g/mol.

In some embodiments, the target compound comprises a compound of formula IV:

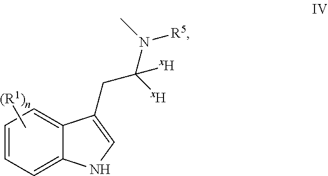

wherein:

each ^{x}H is independently selected from protium and deuterium;

n is selected from 0, 1, 2, 3 and 4;

each R¹ is independently selected from —R³, —OH, —OR³, —O(CO)R³, monohydrogen phosphate, —F, —Cl, —Br and —I; and

each R³ is independently selected from C₁-C₄ alkyl; R⁵ is protium or methyl; and

the compound of formula IV and the compound of formula III differ from one another only by the number of deuterium atoms.

Given that the compound of formula IV and the compound of formula III differ from one another only by the number of deuterium atoms, n and R¹ of both compounds are the same. For the avoidance of doubt, when the target compound is of formula IV, the embodiments described above in which n and R¹ of compounds of formula III are defined must also define n and R¹ of compounds of formula IV.

As described above, to be particularly suitable as an internal standard, the target compound and internal standard should have a similar extraction recovery. In view of the kinetic isotope effect, it is expected that the deuterium atoms of the deutero-monomethylamino or deutero-dimethylamino groups of formula III would reduce the rate of metabolism of the compounds of formula III in plasma with respect to the rate of metabolism of the target compound of formula IV. However, the inventors have found that the intrinsic clearance of compounds of formula IV and their N,N-(dimethyl-d₆) or N-(monomethyl-d₃) analogues are surprisingly similar, indicating that the rate of metabolism of compounds of formula IV and their N,N-(dimethyl-d₆) or N-(monomethyl-d₃) analogues in plasma is also similar. Accordingly, compounds of formula III are particularly useful internal standards for quantifying the amounts of analogous compounds comprising protio-N,N-dimethyl or protio-N,N-monomethyl groups in a sample.

In some embodiments, R⁵ is methyl. Given that the compound of formula IV and the compound of formula III differ from one another only by the number of deuterium atoms, when R⁵ is methyl, R⁴ is —CD₃. Thus, in these embodiments, the compound of formula III comprises a N,N-(dimethyl-d₆) group and the compound of formula IV comprises a N,N-(dimethyl) group.

In particular embodiments, when R⁵ is methyl, the compound of formula IV has a mean molecular weight that is 5.5 to 6.5 g/mol less than the mean molecular weight of the compound of formula III.

The mean molecular weight means the weighted average of molecular weights of the compound of formula III or the compound of formula IV, as measured by an appropriate mass spectroscopic technique, for example LC-MS SIM. In some embodiments, the mean molecular weight is the weighted average. As described above, compounds that are substituted with deuterium may be enriched with deuterium by an amount that is dependent on the percentage of deuterium available in the reagents from which the compounds are derived. For example, not all compounds of formula III comprise d₆-dimethylamino or d₃-monomethylamino substituents—some may comprise do-d₅dimethylamino or do-d₃-monomethylamino substituents.

In some embodiments, R⁵ is protium (thus R⁴ is also protium). In these embodiments, the compound of formula III comprises a N-(methyl-d₃) group and the compound of formula IV comprises a N-(methyl) group.

In particular embodiments, when R⁵ is protium, the compound of formula IV has a mean molecular weight that is 2.5 to 3.5 g/mol less than the mean molecular weight of the compound of formula III.

Typically, the compound of formula IV and the compound of formula III differ from one another only by the number of deuterium atoms on the N,N-(dimethyl) or N-(methyl) group. In other words, ^{x}H of the compound of formula IV and ^{x}H of the the compound of formula III are typically the same. For example, the compound of formula IV may be α-monodeutero-N,N-dimethyltryptamine and the compound of formula III may be α-monodeutero-N,N-(dimethyl-d₆)tryptamine, or the compound of formula IV may be α-monodeutero-N-methyltryptamine and the compound of formula III may be α-monodeutero-N-(methyl-d₃)tryptamine.

In some embodiments, at least one ^{x}H of the compound of formula IV is D. In some embodiments, one ^{x}H of the compound of formula IV is H and the other is D. In other embodiments, each ^{x}H of the compound of formula IV is H. In other embodiments, each ^{x}H of the compound of formula IV is D.

In some embodiments, at least one ^{x}H of the compound of formula III is D. In some embodiments, one ^{x}H of the compound of formula III is H and the other is D. In other embodiments, each ^{x}H of the compound of formula III is H. In other embodiments, each ^{x}H of the compound of formula III is D.

As described above, the invention provides in its seventh aspect the use of a compound of formula III as an internal standard in an assay for quantifying the amount of a target compound in a sample. In some embodiments, the sample comprises a compound of formula I and a compound of formula II as defined in the first aspect, wherein n of compound I is 1, 2, 3 or 4, for example 1, when each ^{x}H is protium, and the compound of formula III and the compound of formula I or the compound of formula II differ only by the number of deuterium atoms they each contain. In these embodiments, the target compound is a compound of formula I or a compound of formula II.

In some embodiments, n of compound I and compound II is 1, 2, 3 or 4, typically 1, when each ^{x}H is protium.

For the avoidance of doubt, embodiments related to the composition of the first aspect, as defined herein, apply mutatis mutandis to these embodiments of the seventh aspect. For example, the composition may comprise a compound of formula I and a compound of formula II in both of which one ^{x}H is H and the other is D; n may be 0, or n may be 1 and R¹ is 5-methoxy; and/or n, and R¹ of both compounds may be the same.

When the compound of formula III and the compound of formula I differ only by the number of deuterium atoms they each contain, R⁴ is —CD₃. In particular embodiments, the compound of formula I has a mean molecular weight that is 5.5 to 6.5 g/mol less than the mean molecular weight of the compound of formula III.

When the compound of formula III and the compound of formula II differ only by the number of deuterium atoms they each contain, R⁴ is protium. In particular embodiments, the compound of formula II has a mean molecular weight that is 2.5 to 3.5 g/mol less than the mean molecular weight of the compound of formula III.

Typically, the compound of formula III and the compound of formula I or the compound of formula II differ from one another only by the number of deuterium atoms on the N,N-(dimethyl) or N-(methyl) group.

The invention provides in its eighth aspect a method of quantifying the amount of a target compound in a sample, the method comprising adding a known amount of a compound of formula III, as defined in the seventh aspect, to the sample.

Embodiments related to the compound of formula III and the target compound of the seventh aspect, as defined herein, apply mutatis mutandis to the eighth aspect. For example, n of the compound of formula III may be 0, or 1 and R¹ may be 5-methoxy; the target compound may comprise a compound of formula IV and the compound of formula IV and formula III differ from one another only by the number of deuterium atoms; and/or the compound of formula IV and the compound of formula III may differ from one another only by the number of deuterium atoms on the N,N-(dimethyl) or N-(methyl) group.

In some embodiments, the sample has been obtained previously from a subject, the target compound having been administered to the subject prior to the sample being obtained. For the avoidance of doubt, neither administration of the target compound to, or obtaining the sample from, the subject are included within the method of the invention although related methods are disclosed herein.

The subject may be any animal, is typically a mammal such as a human.

In some embodiments, the sample is obtained from the blood or urine of the subject. Typically, the sample is plasma of the subject.

In some embodiments, a plurality of samples has been obtained from the subject at different times following administration of the target compound and the method comprises adding a known amount of a compound of formula III to each of the samples and quantifying the amount of target compound in each of the samples.

The amount of target compound is typically quantified by mass spectrometry such as ESI. The target compound may be separated from the other components of the sample using a chromatographic technique such as liquid chromatography, e.g. HPLC, before the amount of target compound is quantified. Signals detected for the target compound of unknown concentration within the samples may be calibrated according to the strength of the signal detected for the compound of formula III of known concentration.

Where a plurality of samples has been obtained from the subject at different times following administration of the target compound, the decrease in concentration of the target compound within the subject over time allows the half-life of the target compound within the subject to be calculated. Thus, in some embodiments, the method further comprises calculating the half-life of the target compound in the subject.

The method of the invention may be used to determine the mean molecular weight, the mean alpha-deuterium saturation and/or the half-life of the target compound within the subject. The target compound may be part of a composition of the invention, having been administered to the subject prior to the sample being obtained. Thus, the method of the invention may be used to characterise the target compounds and/or compositions of the invention

In a further aspect of the invention, there is provided use of a chemical library comprising a plurality of compositions of the first or second aspects and/or target compounds of the seventh aspect for the preparation of a drug substance having a particular pharmacokinetic and/or pharmacodynamic profile, wherein each composition and target compound is characterised by mean molecular weight, mean alpha-deuterium saturation and/or half-life within a subject. In another aspect, there is provided a method of preparing a drug substance having a particular pharmacokinetic or pharmacodynamic profile comprising contacting, e.g. combining, one or more compositions of the first or second aspects and/or target compounds of the seventh aspect, wherein each composition and target compound is characterised by mean molecular weight, mean alpha-deuterium saturation and/or half-life within a subject.

The invention provides in its ninth aspect a compound of formula Ill as defined in the seventh aspect, wherein n is 1, 2, 3 or 4, typically 1, when each ^{x}H is protium.

For the avoidance of doubt, embodiments related to the compound of formula Ill of the seventh aspect, as defined herein, apply mutatis mutandis to the ninth aspect. For example, n of the compound of formula Ill may be 0, or 1 and R¹ may be 5-methoxy; and/or at least one ^{x}H of the compound of formula Ill may be D.

Compounds of formula Ill may be prepared by following the procedures shown in Schemes 1 to 3, discussed above, and replacing dimethylamine and methylamine with d₆-dimethylamine and d₃-methylamine.

Each and every reference referred to herein is hereby incorporated by reference in its entirety, as if the entire content of each reference was set forth herein in its entirety.

The invention may be further understood with reference to examples that follow.

### EXAMPLES

### Synthesis of DMT (SPL026)

### Stage 1: Coupling of indole-3-acetic Acid and Dimethylamine

To a 5 L vessel under N₂ was charged indole-3-acetic acid (257.0 g, 1.467 mol), Hydroxybenzotriazole (HOBt) (˜20% wet) (297.3 g, 1.760 mol) and dichloromethane (DCM) (2313 mL) to give a milky white suspension. Ethylcarbodiimide hydrochloride (EDC.HCl) (337.5 g, 1.760 mol) was then charged portion-wise over 5 minutes at 16-22° C. The reaction mixture was stirred for 2 hours at ambient temperature before 2 M dimethylamine in tetrahydrofuran (THF) (1100 mL, 2.200 mol) was charged dropwise over 20 minutes at 20-30° C. The resultant solution was stirred at ambient temperature for 1 hour where HPLC indicated 1.1% indole-3-acetic acid and 98.1% stage 1. The reaction mixture was then charged with 10% K₂CO₃ (1285 mL) and stirred for 5 minutes. The layers were separated, and the upper aqueous layer extracted with DCM (643 mL×2). The organic extracts were combined and washed with saturated brine (643 mL). The organic extracts were then dried over MgSO₄, filtered and concentrated in vacuo at 45° C. This provided 303.1 g of crude stage 1 as an off-white sticky solid. The crude material was then subjected to a slurry in methyl-t-butyl ether (TBME) (2570 mL) at 50° C. for 2 hours before being cooled to ambient temperature, filtered and washed with TBME (514 mL×2). The filter-cake was then dried in vacuo at 50° C. to afford stage 1 266.2 g (yield=90%) as an off-white solid in a purity of 98.5% by HPLC and >95% by NMR.

### Stage 2: Preparation of DMT

To a 5 L vessel under N₂ was charged stage 1 (272.5 g, 1.347 mol) and THF (1363 mL) to give an off-white suspension. 2.4 M LiAlH₄ in THF (505.3 mL, 1.213 mol) was then charged dropwise over 35 minutes at 20-56° C. to give an amber solution. The solution was heated to 60° C. for 2 hours where HPLC indicated stage 1 ND, stage 2 92.5%, Impurity 1 2.6%, Impurity 2 1.9%. The complete reaction mixture was cooled to ambient temperature and then charged to a solution of 25% Rochelle's salts (aq.) (2725 mL) dropwise over 30 minutes at 20-30° C. The resultant milky white suspension was allowed to stir at 20-25° C. for 1 hour after which the layers were separated and the upper organic layer washed with saturated brine (681 mL). The organic layer was then dried over MgSO₄, filtered and concentrated in vacuo at 45° C. The resultant crude oil was subjected to an azeotrope from ethanol (545 mL×2). This provided 234.6 g (yield=92%) of stage 2 in a purity of 95.0% by HPLC and >95% by NMR.

### Stage 3a (i)-(iii): Preparation of Seed Crystals of DMT Fumarate

(i) Stage 2 (100 mg) was taken up in 8 volumes of isopropyl acetate and warmed to 50° C. before charging fumaric acid (1 equivalent) as a solution in ethanol. The flask was then allowed to mature at 50° C. for 1 hour before cooling to room temperature and stirring overnight, resulting in a white suspension. The solids were isolated by filtration and dried for 4 hours at 50° C. to provide 161 mg of product (>99% yield). Purity by HPLC was determined to be 99.5% and by NMR to be >95%.

(ii) Substitution of isopropyl acetate for isopropyl alcohol in method (i) afforded a white suspension after stirring overnight. The solids were isolated by filtration and dried for 4 hours at 50° C. to provide 168 mg of product (>99% yield). Purity by HPLC was determined to be 99.8% and by NMR to be >95%.

Substitution of isopropyl acetate for tetrahydrofuran in method (i) afforded a white suspension after stirring overnight. The solids were isolated by filtration and dried for 4 hours at 50° C. to provide 161 mg of product (>99% yield). Purity by HPLC was determined to be 99.4% and by NMR to be >95%.

Analysis by x-ray powder diffraction, showed the products of each of methods 9i) to (iii) to be the same, which was labelled Pattern A.

### Stage 3b: Preparation of DMT Fumarate

To a 5 L flange flask under N₂ was charged fumaric acid (152.7 g, 1.315 mol) and Stage 2 (248.2 g, 1.315 mol) as a solution in ethanol (2928 mL). The mixture was heated to 75° C. to give a dark brown solution. The solution was polish filtered into a preheated (80° C.) 5 L jacketed vessel. The solution was then cooled to 70° C. and seeded with Pattern A (0.1 wt %), the seed was allowed to mature for 30 minutes before cooling to 0° C. at a rate of 5° C./hour. After stirring for an additional 4 hours at 0° C., the batch was filtered and washed with cold ethanol (496 mL×2) and then dried at 50° C. overnight. This provided 312.4 g (yield=78%) of Stage 3 in a purity of 99.9% by HPLC and >95% by NMR. XRPD: Pattern A.

### Synthesis of 5MeO-DMT

### Stage 1: Coupling of 5-methoxyindole-3-acetic Acid and Dimethylamine

To a 100 mL 3-neck flask under N₂ was charged 5-methoxyindole-3-acetic acid (3.978 g, 19.385 mmol), HOBt (˜20% wet) (3.927 g, 23.261 mmol) and DCM (40 mL). EDC.HCl (4.459 g, 23.261 mmol) was then charged in portions over 15 minutes at <30° C. The reaction mixture was stirred at ambient temperature for 1 hour before being charged with 2 M dimethylamine (14.54 mL, 29.078 mmol) dropwise over 15 minutes at <25° C. After stirring for 1 hour HPLC indicated no starting material (SM, i.e. 5-methoxyindole-3-acetic acid) remained. The reaction mixture was then charged with 10% K₂CO₃ (20 mL), stirred for 5 minutes then allowed to separate. The lower aqueous layer was removed and back extracted with DCM (10 mL×2). The organic extracts were combined, washed with saturated brine (10 mL) then dried over MgSO₄ and filtered. The filtrate was concentrated in vacuo at 45° C. to provide 3.898 g active (yield=87%) of product in a purity of 95.7% by HPLC.

### Stage 2: Preparation of 5MeO-DMT

To a 100 mL 3-neck flask under N₂ was charged stage 1 methoxy derivative (3.85 g, 16.586 mmol) and THF (19.25 mL). 2.4 M LiAlH₄ in THF (6.22 mL, 14.927 mmol) was then charged dropwise over 30 minutes at <40° C. The reaction mixture was heated to 60° C. for 1 hour where HPLC indicated 0.1% SM (stage 1 methoxy derivative) remained. The reaction mixture was then cooled to ambient temperature and quenched into 25% Rochelle's salts (38.5 mL) dropwise over 30 minutes at <30° C. The resultant suspension was stirred for 1 hour before being allowed to separate. The lower aqueous layer was then removed, and the upper organic layer washed with saturated brine (9.6 mL). The organics were then dried over MgSO₄, filtered and concentrated in vacuo before being subjected to an azeotrope from EtOH (10 mL×2). This provided 3.167 g active (yield=88%) of product in a purity of 91.5% by HPLC.

### Stage 3: Preparation of 5MeO-DMT Fumarate

To a 50 mL 3-neck flask under N₂ was charged fumaric acid (1.675 g, 14.430 mmol) and a solution of stage 2 methoxy derivative (3.15 g, 14.430 mmol) in EtOH (37.8 mL). The mixture was then heated to 75° C. for 1 hour, this did not produce a solution as expected, the mixture was further heated to reflux (78° C.) which still failed to provide a solution. The suspension was therefore cooled to 0-5° C., filtered and washed with EtOH (8 mL×2) before being dried at 50° C. overnight. This provided 3.165 g (yield=65%) of material in a purity of 99.9% by HPLC.

### Synthesis of α,α-dideutero-5-Methoxydimethyltryptamine

For stage 1 (coupling of 5-methoxyindole-3-acetic acid and dimethylamine), see above.

### Stage 2: Preparation of α,α-dideutero-5-Methoxydimethyltryptamine

To a 100 mL 3-neck flask under N₂ was charged stage 1 methoxy derivative (3.85 g, 16.586 mmol) and THF (19.25 mL). 2.4 M LiAlD₄ in THF (6.22 mL, 14.927 mmol) was then charged dropwise over 30 minutes at <40° C. The reaction mixture was heated to 60° C. for 1 hour where HPLC indicated 0.1% SM (stage 1 methoxy derivative) remained. The reaction mixture was then cooled to ambient temperature and quenched into 25% Rochelle's salts (38.5 mL) dropwise over 30 minutes at <30° C. The resultant suspension was stirred for 1 hour before being allowed to separate. The lower aqueous layer was then removed, and the upper organic layer washed with saturated brine (9.6 mL). The organics were then dried over MgSO₄, filtered and concentrated in vacuo before being subjected to an azeotrope from EtOH (10 mL×2). This provided 3.196 g active (yield=88%) of product in a purity of 91.5% by HPLC.

### Stage 3: Preparation of α,α-dideutero-5-Methoxydimethyltryptamine Fumarate

To a 50 mL 3-neck flask under N₂ was charged fumaric acid (1.675 g, 14.430 mmol) and a solution of stage 2 methoxy derivative (3.15 g, 14.430 mmol) in EtOH (37.8 mL). The mixture was then heated to 75° C. for 1 hour, this did not produce a solution as expected, the mixture was further heated to reflux (78° C.) which still failed to provide a solution. The suspension was therefore cooled to 0-5° C., filtered and washed with EtOH (8 mL×2) before being dried at 50° C. overnight. This provided 3.165 g (yield=65%) of material in a purity of 99.9% by HPLC.

### Synthesis of Deuterated Mixtures of DMT Compounds (SPL028i to SPL028vi)

A modified synthesis at stage 2 using solid LiAlH₄/LiAlD₄ mixtures was adopted, using 1.8 equivalents of LiAlH₄/LiAlD₄ versus 0.9 equivalents using the process described above for undeuterated DMT.

Six deuteration reactions were performed.

Representative Synthesis of a Deuterated Mixture (Using 1:1 LiAlH₄:LiAlD₄) of DMT Compounds

To a 250 mL 3-neck flask under N₂ was charged LiAlH₄ (1.013 g, 26.7 mmol), LiAlD₄ (1.120 g, 26.7 mmol) and THF (100 mL). The resultant suspension was stirred for 30 minutes before stage 1 (6 g, 29.666 mmol) was charged portion-wise over 15 minutes at 20-40° C. The reaction mixture was then heated to reflux (66° C.) for 2 hours where HPLC indicated no stage 1 remained. The mixture was cooled to 0° C. and quenched with 25% Rochelle's salts (aq) (120 mL) over 30 minutes at <30° C. The resultant milky suspension was stirred for 1 hour and then allowed to separate. The lower aqueous layer was removed and the upper organic layer washed with saturated brine (30 mL). The organics were then dried over MgSO₄, filtered and concentrated in vacuo. This provided 4.3 g of crude material. The crude was then taken up in ethanol (52 mL) and charged with fumaric acid (2.66 g, 22.917 mmol) before heating to 75° C. The resultant solution was allowed to cool to ambient temperature overnight before further cooling to 0-5° C. for 1 hour. The solids were isolated by filtration and washed with cold ethanol (6.5 mL×2). The filtercake was dried at 50° C. overnight to provided 5.7 g (yield=63%) of product in a purity of 99.9% by HPLC and >95% by NMR.

Synthesis of d₆-DMT (SPL028vii)

### Stage 1

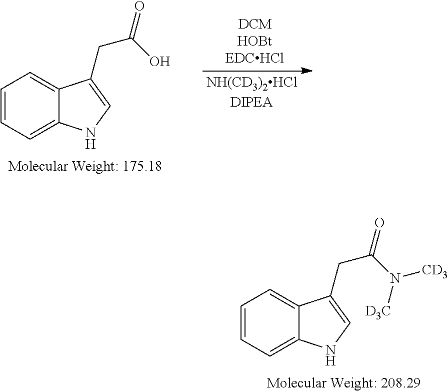 [SMILES: O=C(O)Cc1c[nH]c2ccccc12 ; [2H]C([2H])([2H])N(C(=O)Cc1c[nH]c2ccccc12)C([2H])([2H])[2H]]

EDC.HCl (15.7 g, 81.90 mmol) was added to 3-indoleacetic acid (12.0 g, 68.50 mmol) and HOBt.H₂O (1.16 g, 75.75 mmol) in DCM (108 mL) at room temperature. The reaction was stirred for 1 hour after which N,N-diisopropylethylamine (DIPEA) (35.6 mL, 205.75 mmol) and d₆-dimethylamine.HCl (9.0 g, 102.76 mmol) were added (temperature maintained below 30° C.). The reaction was stirred for 1 hour at room temperature after which analysis by HPLC indicated 65.6% product with 28.9% 3-indoleacetic acid remaining. DIPEA (11.9 mL, 68.78 mmol) was added and the reaction was stirred for 1 hour at room temperature. HPLC indicated no change in conversion. Aqueous potassium carbonate (6.0 g in 54 mL water) was added and the phases were separated. The aqueous phase was extracted with DCM (2×30 mL). The combined organics were washed with brine (2×30 mL) then aqueous citric acid (20 w/w %, 50 mL), dried over MgSO₄ and filtered. The filtrate was stripped and the resulting solids were slurried in TBME (120 mL) and isolated by filtration. Purification by flash column chromatography yielded 8.34 g of the desired product (58% yield). ¹H NMR confirmed the identity of the product.

### Stage 2

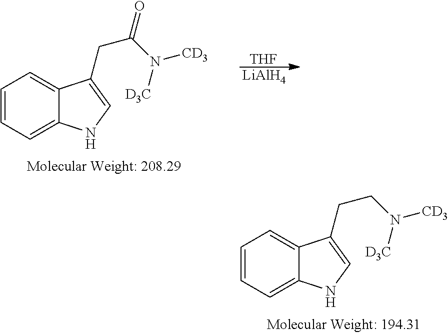 [SMILES: [2H]C([2H])([2H])N(C(=O)Cc1c[nH]c2ccccc12)C([2H])([2H])[2H] ; "LiAlH4" ; [2H]C([2H])([2H])N(CCc1c[nH]c2ccccc12)C([2H])([2H])[2H] ; "THF"]

LiAlH₄ (1 M in THF, 17.3 mL, 17.28 mmol) was added to a suspension of stage 1 (4.0 g, 19.20 mmol) in THF (10 mL) at <30° C. The resulting reaction was heated to 60-65° C. and stirred for 2 hours. HPLC analysis indicated complete consumption of stage 1 with 97.3% product formed. The reaction was cooled to room temperature and quenched into aqueous Rochelle's salts (10 g in 30 mL water) at <30° C. After stirring for 1 hour, the phases were separated. The aqueous phase was extracted with THF (20 mL). The combined organics were washed with brine (20 mL), dried over MgSO₄, filtered and stripped (azeotroped with ethanol, 20 mL) to give the desired product as an amber oil (3.97 g). ¹H NMR confirmed the identity of the product and indicated 8.5% ethanol was present (no THF) giving an active yield of 3.63 g, 97%.

### Stage 3

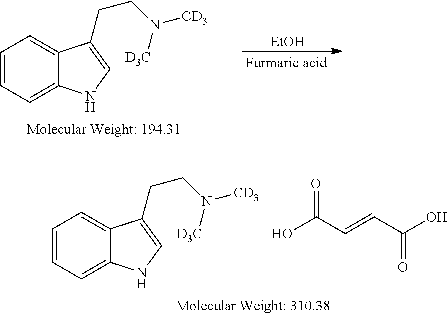 [SMILES: "EtOH" ; [2H]C([2H])([2H])N(CCc1c[nH]c2ccccc12)C([2H])([2H])[2H] ; [2H]C([2H])([2H])N(CCc1c[nH]c2ccccc12)C([2H])([2H])[2H] ; O=C(O)/C=C/C(=O)O]

d₆-DMT free base (3.6 g active, 18.53 mmol) was dissolved in ethanol (43 mL) at room temperature. Fumaric acid (2.15 g, 18.53 mmol) was added and the solution was heated to 75° C. (solids crystallised during heating and did not re-dissolve). The resulting suspension was cooled to 0-5° C. and stirred for 1 hour. The solids were isolated by filtration, washed with ethanol (2×7 mL) and pulled dry. Further drying in a vacuum oven at 50° C. yielded the desired d₆-DMT fumaric acid salt (4.98 g, 87%).

Synthesis of d₈-DMT (SPL028viii)

For stage 1 (coupling of 3-indoleacetic acid and d₆-dimethylamine), see above

### Stage 2

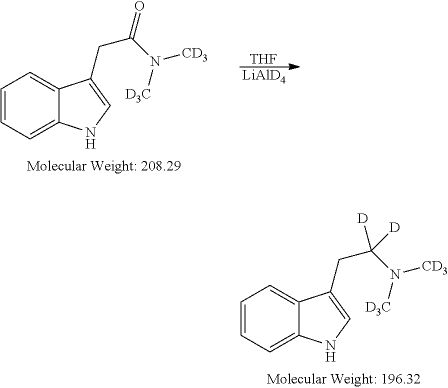

LiAlD₄ (1 M in THF, 17.3 mL, 17.28 mmol) was added to a suspension of stage 1 (4.0 g, 19.20 mmol) in THF (10 mL) at <30° C. The resulting reaction was heated to 60-65° C. and stirred for 2 hours. HPLC analysis indicated complete consumption of the stage 1 with 97.3% product formed. The reaction was cooled to room temperature and quenched into aqueous Rochelle's salts (10 g in 30 mL water) at <30° C. After stirring for 1 hour, the phases were separated. The aqueous phase was extracted with THF (20 mL). The combined organics were washed with brine (20 mL), dried over MgSO₄, filtered and stripped (azeotroped with ethanol, 20 mL) to give the desired product as an amber oil (4.01 g). ¹H NMR confirmed the identity of the product and indicated 8.6% ethanol was present (no THF) giving an active yield of 3.66 g, 97%.

### Stage 3

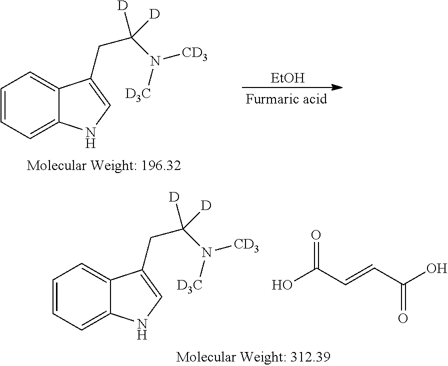 [SMILES: "EtOH" ; [2H]C([2H])([2H])N(C([2H])([2H])[2H])C([2H])([2H])Cc1c[nH]c2ccccc12 ; [2H]C([2H])([2H])N(C([2H])([2H])[2H])C([2H])([2H])Cc1c[nH]c2ccccc12 ; O=C(O)/C=C/C(=O)O]

d₈-DMT free base (3.6 g active, 18.53 mmol) was dissolved in ethanol (43 mL) at room temperature. Fumaric acid (2.15 g, 18.53 mmol) was added and the solution was heated to 75° C. (solids crystallised during heating and did not re-dissolve). The resulting suspension was cooled to 0-5° C. and stirred for 1 hour. The solids were isolated by filtration, washed with ethanol (2×7 mL) and pulled dry. Further drying in a vacuum oven at 50° C. yielded the desired d₈-DMT fumaric acid salt (4.62 g, 81%).

### Assessment of Extents of Deuteration

This was achieved by LCMS-SIM (SIM=single ion monitoring), the analysis giving a separate ion count for each mass for the deuterated N,N-dimethyltryptamine compounds at the retention time for N,N-dimethyltryptamine. The percentage of each component was then calculated from these ion counts.

For example, % D0=[D0/(D0+D1+D2)]×100.

### HPLC Parameters

System: Agilent 1100/1200 series liquid chromatograph or equivalent

| Column: | Triart Phenyl; 150 × 4.6 mm, 3.0 μm particle size | | |
|---|---|---|---|
| | (Ex: YMC, Part number: TPH12S03-1546PTH) | | |
| Mobile phase A: | Water:Trifluoroacetic acid (100:0.05%) | | |
| Mobile phase B: | Acetonitrile:Trifluoroacetic acid (100:0.05%) | | |
| Gradient: | Time | % A | % B |
| | 0 | 95 | 5 |
| | 13 | 62 | 38 |
| | 26 | 5 | 95 |
| | 30.5 | 5 | 95 |
| | 31 | 95 | 5 |
| Flow rate: | 1.0 mL/min | | |
| Stop time: | 31 minutes | Post runtime: | 4 minutes |
| Injection volume: | 5 μL | Wash vial: | N/A |
| Column | 30° C. | | |
| temperature: | combined | | |
| Wavelength: | 200 nm, (4 nm) | Reference: | N/A |

### Mass Spectrometry Parameters

System: Agilent 6100 series Quadrupole LC-MS or equivalent
Drying gas flow: 12.0 L/min Drying gas temp.: 350° C.
Nebuliser pressure: 35 psig
Fragmentor: 110 Gain: 1.00

| Cpd | RT | RRT | Conc | Diluent | Detection | Mass |
|---|---|---|---|---|---|---|
| D0 | 10.64 | 1.00 | 0.30 mg/ml | CH₃CN:H₂O (50:50) | (+) SIM | 189.10 m/z |
| D1 | 10.64 | 1.00 | 0.30 mg/ml | CH₃CN:H₂O (50:50) | (+) SIM | 190.10 m/z |
| D2 | 10.64 | 1.00 | 0.30 mg/ml | CH₃CN:H₂O (50:50) | (+) SIM | 191.10 m/z |

MS-SIM ramie is the target mass ±0.1 m/z

| | Weighted | | | | | | | | |
|---|---|---|---|---|---|---|---|---|---|
| Compound | MW | | | | | | | | |
| Name | (g/mol) | D₀ | D₁ | D₂ | D₄ | D₅ | D₆ | D₇ | D₈ |
| SPL026 | 188.27 | 0 | 0 | 0 | 0 | 0 | 0 | 0 | 0 |
| SPL028i | 190.2398 | 0.70% | 2.70% | 96.60% | 0 | 0 | 0 | 0 | 0 |
| SPL028ii | 189.1915 | 30.00% | 48.30% | 21.70% | 0 | 0 | 0 | 0 | 0 |
| SPL028iii | 189.6685 | 16.50% | 46.80% | 36.80% | 0 | 0 | 0 | 0 | 0 |
| SPL028iv | 189.6764 | 9.30% | 41.50% | 49.20% | 0 | 0 | 0 | 0 | 0 |
| SPL028v | 188.9098 | 47.50% | 41.30% | 11.20% | 0 | 0 | 0 | 0 | 0 |
| SPL028vi | 188.9613 | 57.40% | 35.30% | 7.40% | 0 | 0 | 0 | 0 | 0 |
| SPL028vii | 194.308 | 0.00% | 0 | 0 | 0.01% | 1.20% | 98.80% | 0 | 0 |
| SPL028viii | 196.318 | 0.00% | 0 | 0 | 0 | 0 | 0.10% | 3.20% | 96.70% |

### In Vitro Intrinsic Clearance of DMT (SPL026) and 8 Deuterated Compound Blends

In vitro determination of intrinsic clearance is a valuable model for predicting in vivo hepatic clearance. The liver is the main organ of drug metabolism in the body, containing both phase I and phase II drug metabolising enzymes, which are present in the intact cell.

### Aim

To use human hepatocytes to assess the in vitro intrinsic clearance of deuterated DMT analogue blends relative to DMT.

### Description of the Experiment

Human (mixed gender) hepatocytes pooled from 10 donors were used to investigate the in vitro intrinsic clearance of DMT and 8 deuterated analogues in three separate experiments:
1) In vitro human hepatic intrinsic clearance of DMT (SPL026) and 6 d₂-DMT analogue blends (SPL028i-vi)
2) In vitro human hepatocyte intrinsic clearance of 6 d₂-DMT analogue blends (SPL028i-vi) and d₆-DMT (SPL028vii)
3) In vitro human hepatocyte intrinsic clearance of DMT, 2 d₂-DMT analogue blends (SPL028i and SPL028ii), d₆-DMT (SPL028vii) and d₈-DMT (SPL028viii)

A concentration of 5 μM was used for all test compounds, as well as sumatriptan, serotonin, benzylamine controls with 2 replicate incubations per compound in each experiment. This concentration was chosen in order to maximise the signal-to-noise ratio, while remaining under the Michaelis constant (Km) for the monoamine oxidase enzyme (MAO). Diltiazem and diclofenac controls were used at a laboratory-validated concentration of 1 μM.

Test compounds were mixed with the hepatocyte suspension within a 96-well plate and incubated for up to 60 minutes at 37° C. The suspension was continuously agitated. At 7 time points, small aliquots were withdrawn, and the test compound/blend concentration therein was measured by LC-MS/MS. The time points measured were 2, 4, 8, 15, 30, 45 and 60 minutes.

The following LC-MS/MS conditions were used for the analysis:

Instrument: Thermo TSQ Quantiva with Thermo Vanquish UPLC system

### Column: Luna Omega 2.1×50 mm 2.6 μm

Solvent A: H₂O+0.1% formic acid
Solvent B: Acetonitrile+0.1% formic acid
Flow rate: 0.8 ml/min

### Injection vol: 1 μl

Column temp: 65° C.

### Gradient:

| Time (mins) | % Solvent B |
|---|---|
| 0.00 | 5.0 |
| 0.90 | 75.0 |
| 1.36 | 99.0 |
| 1.36 | 5.0 |
| 1.80 | 5.0 |

MS parameters:

| | |
|---|---|
| Positive ion spray voltage: | 4000 V |
| Vaporiser temperature: | 450° C. |
| Ion transfer tube temp: | 365° C. |
| Sheath gas: | 54 |
| Aux gas: | 17 |
| Sweep gas: | 1 |
| Dwell time | 8 ms |

MRM transitions:
D0=mass to charge ratio 189.14>58.16/144.179
D1=mass to charge ratio 190.14>59.17
D2=mass to charge ratio 191.14>60.17/146.177
D6=mass to charge ratio 195.17>64.127
D8=mass to charge ratio 197.2>146.17

The MRM transitions were determined from a preliminary analysis of DMT samples containing either no deuterium (for D0 transition), or high levels of either D1, D2, D6 or D8 deuteration (for the D1, D2, D6 and D8 transitions respectively).

The resulting concentration-time profile was then used to calculate intrinsic clearance (CLint) and half-life (t½). To do this, the MS peak area or MS peak area/IS response of each analyte is plotted on a natural log scale on the y-axis versus time (min) of sampling on the X-axis. The slope of this line is the elimination rate constant. This is converted to a half-life by −ln(2)/slope. Intrinsic clearance is calculated from the slope/elimination rate constant and the formula is CLint=(−1000*slope)/cell density in 1E6 cells/ml, to give units of microlitre/min/million cells.

### Results

1) In vitro human hepatic intrinsic clearance of DMT (SPL026) and 6 d₂-DMT analogue blends (SPL028i-vi)

Intrinsic clearance and half-life values were calculated for DMT and the 6 deuterated mixtures described above. These data were weighted dependent on the ratio of DO, D1 and D2 to give an overall intrinsic clearance and half-life value for each compound blend (Table 2).

TABLE 2
In vitro intrinsic clearance and half-life of SPL026 and 6 different d₂-
deuterated SPL028 analogue blends in human hepatocytes

| | | | Intrinsic | |
|---|---|---|---|---|
| | | Percentage | clearance | |
| | Molecular | ratio of | (μL/min/ | Half-life |
| Compound | weight | D₂ (%) | million cells) | (min) |
| SPL026 | 188.269 | 0 | 13.77 | 92.39 |
| SPL028i | 190.240 | 96.60 | 7.15 | 178.79 |
| SPL028ii | 189.192 | 21.70 | 10.46 | 125.80 |
| SPL028iii | 189.669 | 36.80 | 9.36 | 140.43 |
| SPL028iv | 189.676 | 49.20 | 11.14 | 116.84 |
| SPL028v | 188.910 | 11.20 | 10.99 | 119.61 |
| SPL028vi | 188.961 | 7.40 | 13.64 | 95.04 |
| Benzylamine | | | 16.7 | 76.3 |
| Serotonin | | | 38.6 | 33.0 |

Data were fitted with a linear model using regression analysis, which revealed that deuterium enrichment at the α-carbon of DMT decreases intrinsic clearance linearly with increasing molecular weight (MW), therefore enabling manufacture of DMT drug substances with half-lives which can be accurately predicted in the range identified.

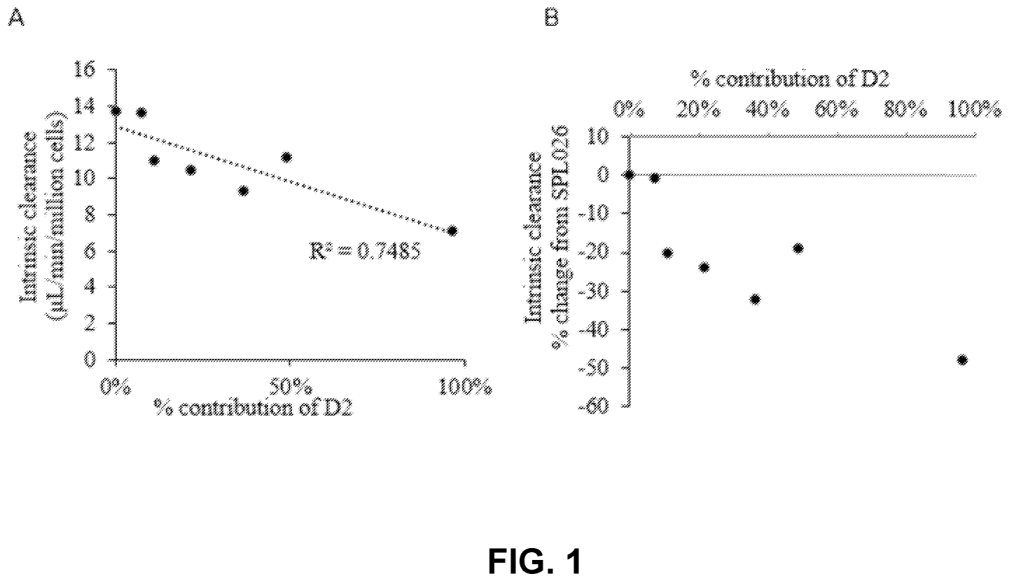

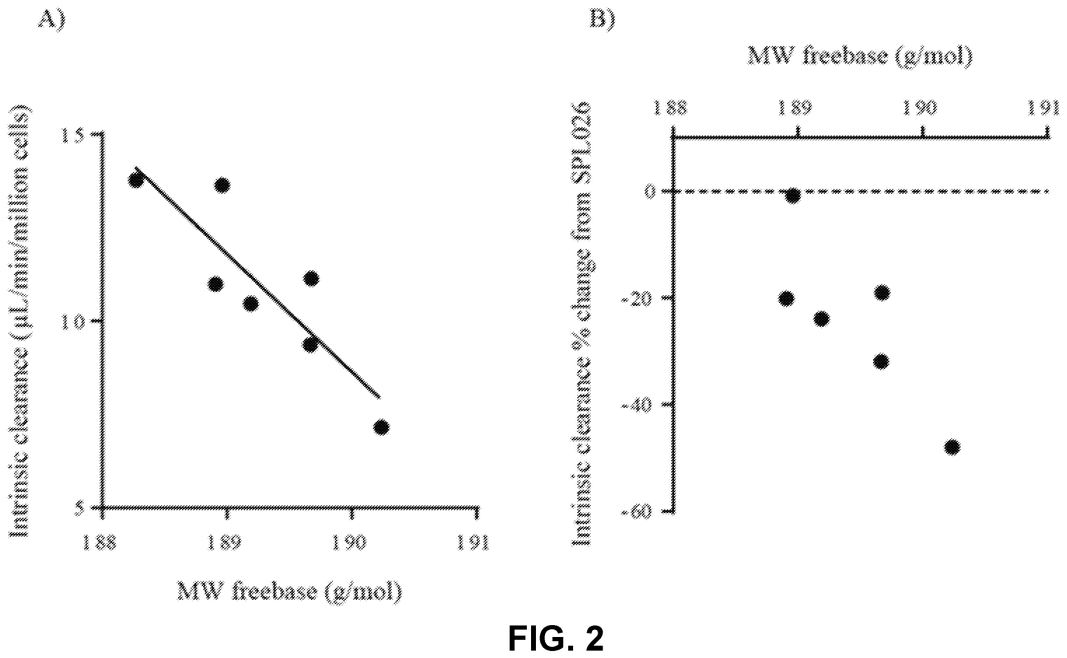

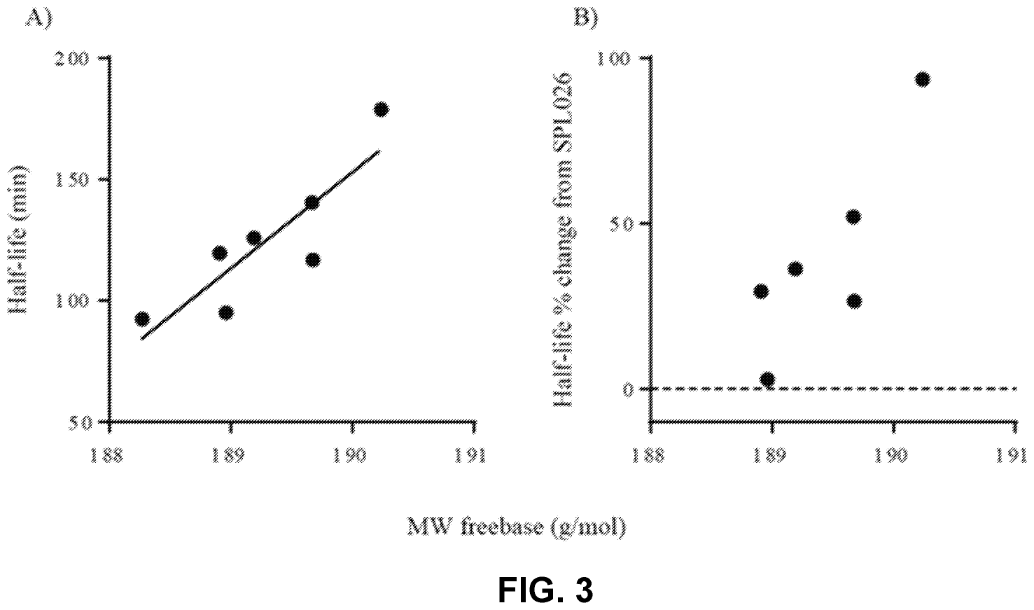

SPL028i, which contains 96.6% D2-DMT, sees the biggest change, with the intrinsic clearance rate almost halved compared to undeuterated-DMT (FIG. 1 and FIG. 2), nearly doubling the half-life (FIG. 3). Intermediate blends of deuteration (Mixtures 2 to 5) decreased intrinsic clearance in a manner correlated with molecular weight (FIG. 2).
2) In vitro human hepatocyte intrinsic clearance of 6 d₂-DMT analogue blends (SPL028i-vi) and d₆-DMT (SPL028vii)

Intrinsic clearance and half-life values were calculated for 6 d₂-DMT analogue blends and d₆-DMT described above (Table 3).

TABLE 3
In vitro intrinsic clearance and half-life of six d₂₋DMT
analogue blends (SPL028i-vi) and
d₆-DMT (SPL028vii) in human hepatocytes

| | Intrinsic clearance | |
|---|---|---|
| | (μL/min/million | |
| Compound Name | cells) | Half-life (min) |
| SPL028i | 6.3 | 258.3 |
| SPL028ii | 9.1 | 191.1 |
| SPL028iii | 8.2 | 213.9 |
| SPL028iv | 7.7 | 223.9 |
| SPL028v | 14.1 | 119.0 |
| SPL028vi | 13.4 | 126.8 |
| SPL028vii (D₆₎ | 13.3 | 122.2 |
| Diltiazem (A) | 15.3 | 15.0 |
| Diltiazem (B) | 17.2 | 18.2 |
| Diclofenac (A) | 155.0 | 154.0 |
| Diclofenac (B) | 150.1 | 154.3 |

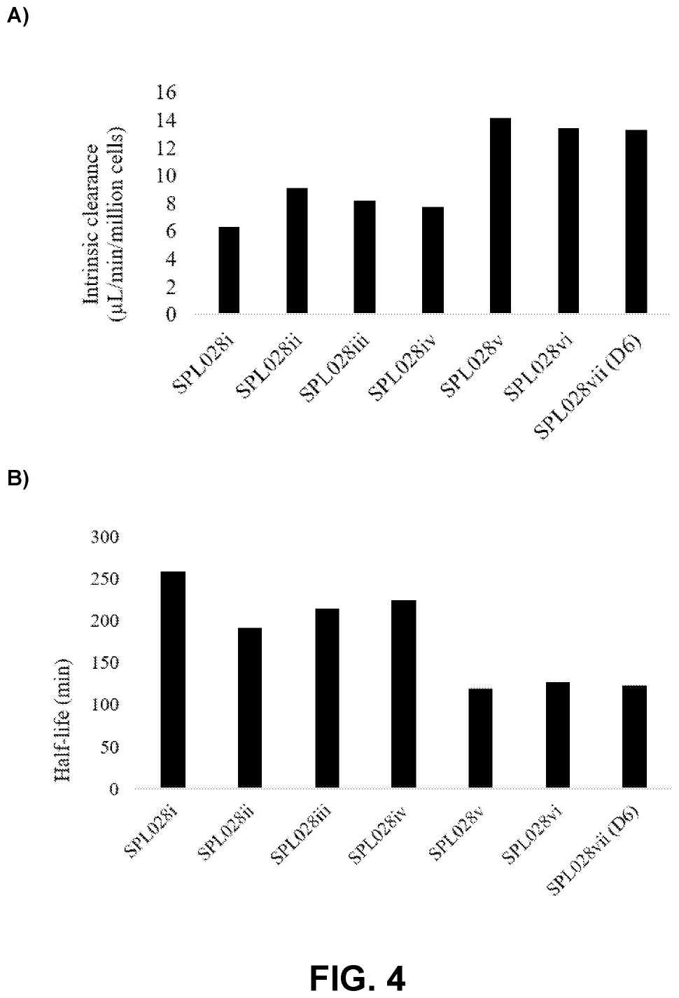

Data were fitted with a linear model using regression analysis, which confirmed the previous findings that that deuterium enrichment at the α-carbon of DMT decreases intrinsic clearance linearly with increasing level of d₂-deuteration. The intrinsic clearance and half-life values of d₆-DMT (SPL028vii) are most similar to those of SPL028vi (the d₂-DMT analogue blend with the least amount of d₂-DMT (7.40%) and the most amount of d₀-DMT (57.40%)) (see FIG. 4). Deuteration at the dimethylamino group has little effect on the intrinsic clearance and half-life of DMT. Consequently, molecular weight is a weak predictor of intrinsic clearance of d₂-deuterated and d₆-deuterated blends.

3) In vitro human hepatocyte intrinsic clearance of 2 d₂-DMT analogue blends (SPL028i and SPL028ii), d₆-DMT (SPL028vii) and d₈-DMT (SPL028viii)

Intrinsic clearance and half-life values were calculated for 2 d₂-DMT analogue blends, d₆-DMT and d₈-DMT analogue blends described above (Table 4).

TABLE 4
In vitro intrinsic clearance and half-life of DMT,
two d₂₋DMT analogue blends (SPL028i and ii), d₆-DMT
(SPL028vii) and d₈-DMT (SPL028viii) in human hepatocytes

| | Intrinsic Clearance | |
|---|---|---|
| | (μL/min/million | |
| Compound Name | cells) | Half-life (min) |
| SPL026 | 19.4 | 98.9 |
| SPL028i | 8.3 | 233.1 |
| SPL028ii | 11.7 | 170.9 |
| SPL028vii | 17.1 | 112.1 |
| SPL028viii | 9.3 | 206.9 |
| Diltiazem | 22.0 | 87.3 |
| Diclofenac | 92.5 | 20.7 |

Data were fitted with a linear model using regression analysis, which confirmed the previous findings that that deuterium enrichment at the α-carbon of DMT decreases intrinsic clearance linearly with increasing level of d₂-deuteration and that molecular weight is a weak predictor of intrinsic clearance.

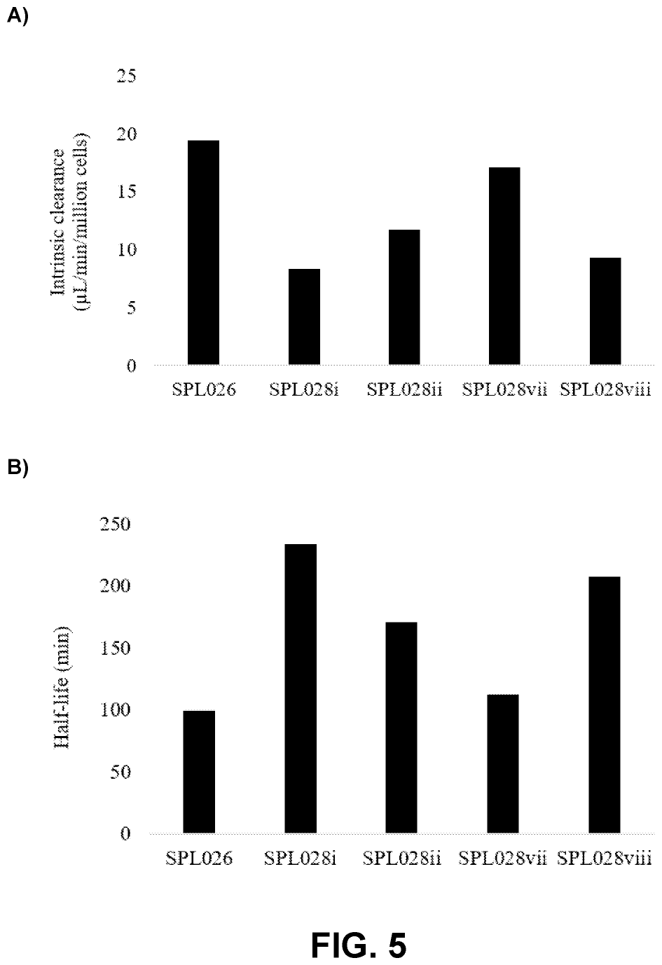

Deuteration at the dimethylamino group in d₆-DMT (SPL028vii) has minimal effect on the intrinsic clearance in human hepatocytes relative to DMT (SPL026) (see FIG. 5). Complete deuterium enrichment at both the α-carbon and dimethylamino group of DMT in d₈-DMT (SPL028viii) did not significantly change the metabolic stability relative to the d₂-DMT blend comprising the most d₂-DMT and the least do-DMT (SPL028i—96.60% D2 and 0.70% DO) (see FIG. 5).

### CONCLUSION

These data demonstrate that increasing deuterium enrichment at the α-carbon of DMT increases metabolic stability, leading to a decrease in clearance and longer half-life. A linear relationship exists between level of d₂-deuteration and half-life. Molecular weight is a weak predictor of intrinsic clearance of d₂-deuterated, d₆-deuterated and d₈-deuterated blends: deuteration of the dimethylamino group of DMT has minimal effect on intrinsic clearance and half-life in human hepatocytes. The relative half-life of analogous mixtures of protio, mono- and di-deutero compounds of NMT and substituted NMT and DMT are expected to mirror the trends observed here. It is expected that increasing deuterium enrichment at the α-carbon of compounds of NMT and substituted NMT and DMT increases metabolic stability, leading to a decrease in clearance and longer half-life. It is expected that deuterating the dimethylamino or methylamino groups of NMT and substituted NMT and DMT has minimal effect on intrinsic clearance and half-life.

1. A composition comprising a compound of formula I and a compound of formula II: 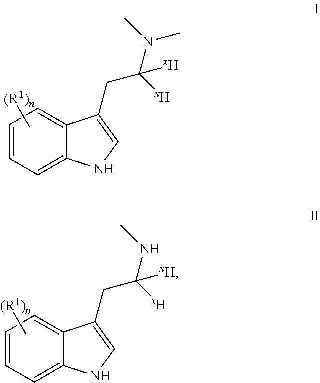
wherein:
each ^{x}H is independently selected from protium and deuterium;
n is selected from 0, 1, 2, 3 and 4;
each R¹ is independently selected from —R³, —OH, —OR³, —O(CO)R³, monohydrogen phosphate, —F, —Cl, —Br and —I; and
each R³ is independently selected from C₁-C₄alkyl.

2. The composition of claim 1, comprising about 5% to about 95% by weight of the compound of formula I.

3. The composition of claim 1, comprising a compound of formula I and a compound of formula II in both of which one ^{x}H is H and the other is D.

4. The composition of claim 1, comprising a compound of formula I and a compound of formula II in both of which each ^{x}H is H.

5. The composition of claim 1, comprising a compound of formula I and a compound of formula II in both of which each ^{x}H is D.

6. The composition of claim 1, wherein R¹ is independently selected from —OR³, —O(CO)R³, monohydrogen phosphate and —OH.

7. The composition of claim 1, wherein R³ is methyl.

8. The composition of claim 1, wherein n is 1.

9. The composition of claim 8 wherein R¹ is at the 4- or 5-position.

10. The composition of claim 1, wherein n is 0, or n is 1 and R¹ is selected from 5-methoxy, 4-acetoxy, 4-monohydrogen phosphate, 4-hydroxy and 5-hydroxy.

11. The composition of claim 1, wherein n is 0, or n is 1 and R¹ is 5-methoxy.

12. The composition of claim 1, comprising a compound of formula I and a compound of formula II in both of which ^{x}H, n, and R¹ are the same.

13. The composition of claim 1, which comprises two compounds of formula I, which differ from one another only by the definition of ^{x}H.

14. The composition of claim 1, which comprises two compounds of formula II, which differ from one another only by the definition of ^{x}H.

15.-23. (canceled)

24. Use of a compound of formula Ill: 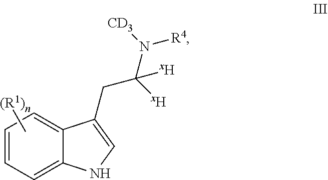
wherein:
each ^{x}H is independently selected from protium and deuterium;
n is selected from 0, 1, 2, 3 and 4;
each R¹ is independently selected from —R³, —OH, —OR³, —O(CO)R³, monohydrogen phosphate, —F, —Cl, —Br and —I; and
each R³ is independently selected from C₁-C₄ alkyl;
R⁴ is protium or —CD₃,
wherein n is 1, 2, 3 or 4 when each ^{x}H is protium and R⁴ is —CD₃,
as an internal standard in an assay for quantifying the amount of a target compound in a sample.

25. The use of claim 24, wherein the target compound comprises a compound of formula IV: 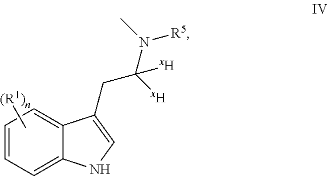
wherein:
each ^{x}H is independently selected from protium and deuterium;
n is selected from 0, 1, 2, 3 and 4;
each R¹ is independently selected from —R³, —OH, —OR³, —O(CO)R³, monohydrogen phosphate, —F, —Cl, —Br and —I; and
each R³ is independently selected from C₁-C₄ alkyl;
R⁵ is protium or methyl; and
the compound of formula IV and the compound of formula III differ from one another only by the number of deuterium atoms.

26. The use of claim 25, wherein R⁵ is methyl and the compound of formula IV has a mean molecular weight that is 5.5 to 6.5 g/mol less than the mean molecular weight of the compound of formula III.

27. The use of claim 25, wherein R⁵ is protium and the compound of formula IV has a mean molecular weight that is 2.5 to 3.5 g/mol less than the mean molecular weight of the compound of formula III.

28.-39. (canceled)

40. A method of treating a psychiatric or neurological disorder in a patient, wherein the psychiatric or neurological disorder is selected from (i) an obsessive compulsive disorder, (ii) a depressive disorder, (iii) a schizophrenia disorder, (iv) a schizotypal disorder, (v) an anxiety disorder, (vi) substance abuse, and (vii) an avolition disorder, the method comprising:
administering to a patient in need thereof a composition of claim 1.

41. A method for identifying an amount of a composition of claim 1 in a sample, comprising:
using a compound of formula Ill: 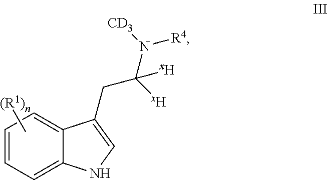
wherein:
each ^{x}H is independently selected from protium and deuterium;
n is selected from 0, 1, 2, 3 and 4;
each R¹ is independently selected from —R³, —OH, —OR³, —O(CO)R³, monohydrogen phosphate, —F, —Cl, —Br and —I; and
each R³ is independently selected from C₁-C₄ alkyl;
R⁴ is protium or —CD₃,
wherein n is 1, 2, 3 or 4 when each ^{x}H is protium and R⁴ is —CD₃,
as an internal standard in an assay for quantifying the amount of composition of claim 1 in the sample, comprising adding a known amount of a compound of formula Ill to the sample and calculating the half-life of the composition of claim 1.
